(19) United States
(12) Patent Application Publication
(10) Pub. No.: US 2023/0041557 A1
(43) Pub. Date: Feb. 9, 2023

(54) NURSERY BASED DEVICES WITH CONNECTIVITY TO IOT ECOSYSTEM

(71) Applicant: MUNCHKIN, INC., Van Nuys, CA (US)

(72) Inventors: Agnes Yena Lee, Los Angeles, CA (US); Sung Yun Chan, Pasadena, CA (US); Nicolas Arthur Trumbo, Valencia, CA (US); Steven Bryan Dunn, Beverly Hills, CA (US); Brian Chi Ho Gum, Northridge, CA (US); Kevin Douglas Johnson, Tarzana, CA (US); Sean Quinton Herrin, South Pasadena, CA (US); Chiu Wa Lau, Hong Kong (CN); Mau Chun Yip, Hong Kong (CN); Britt Ashcraft, Valencia, CA (US); Robert Zacaries Evora, Simi Valley, CA (US)

(21) Appl. No.: 17/881,530

(22) Filed: Aug. 4, 2022

Related U.S. Application Data
(60) Provisional application No. 63/237,476, filed on Aug. 26, 2021.
(60) Provisional application No. 63/229,450, filed on Aug. 4, 2021.

(51) Int. Cl. A61J 17/00; G02B 26/00; F21V 14/08; F24F 11/00; A61N 5/06; A61M 21/02; B01D 46/44; B01D 53/04; B01D 53/86

(52) U.S. Cl. CPC A61J 17/1012; G02B 26/008; F21V 14/08; F24F 11/0008; A61J 17/1011; A61N 5/0618; A61M 21/02; B01D 46/442; B01D 53/0454; B01D 53/8696; A61N 2005/0663; A61N 2005/0651; A61M 2021/0027

(57) ABSTRACT

The present disclosure presents infant soothing devices which provide timed, color, and intensity controlled light and sound projections to assist in sleep therapy. The devices may be part of a smart nursery which has a number of electronically connected devices which communicate with a central electronic application to gather information and provide operation instructions to the devices. The smart nursery may be connected to a larger network to provide feedback and controllability outside of the nursery.

### CROSS REFERENCE TO RELATED APPLICATION

This Patent application claims priority to U.S. Provisional Patent Application Ser. No. 63/237,476, filed Aug. 26, 2021, and to U.S. Provisional Patent Application Ser. No. 63/229,450, filed Aug. 4, 2021; the contents of which are hereby incorporated by reference herein in their entirety into this disclosure.

### TECHNICAL FIELD

The subject disclosure relates generally to a sleep training and light therapy devices. More specifically, the subject disclosure relates to nursery based devices with connectivity to an IoT ecosystem.

### BACKGROUND OF THE SUBJECT DISCLOSURE

Proper care for children is a parent's most important task. At every stage in a child's life, a parent strives to watch over, protect, and properly care for the child. When a child is a newborn or infant, such proper care is even more urgent as the child has not yet learned to communicate. It is universally accepted that newborns and young children have not yet developed a circadian rhythm that allow a routine wake and sleep cycle. Thus, the lack of a proper sleep cycle has a strenuous effect on parents and those who watch such infants as the caretakers must also essentially match the lack of proper sleep cycle of the infant. Thus, parents and caretakers have much incentive to train an infant or young child to properly sleep so that their sleep/awake cycles coincide with the dark and light circadian cycles. One way to watch over the infant or child during such sleep training is through audio or video monitors.

There are numerous products in the market that serve to assist a parent in monitoring their child, either audibly or visually, from a separate location. Such audio and video monitors have become a standard tool that parents now use to ensure that their child is safe even if the parent is in a different physical location than the child. As helpful as these audio and visual monitors have become, they still do not provide the parent with all the information needed to ensure that their child is safe from inaudible or invisible threats to their safety, particularly when the infant or child is sleeping. Such threats can be harmful gases or non-ideal air conditions in the child's room.

### SUMMARY OF THE SUBJECT DISCLOSURE

The present subject disclosure presents a soother for sleep training and light therapy for infants and small children. The soother may be a standalone device and/or part of a larger IoT network of smart devices for assisting parents with childcare and nursery management. The IoT network may include a humidifier with at least one accompanying sensor and an air purifier with at least one accompanying sensor. The IoT network may include a monitor application for smart devices, and can be part of a larger network of connected smart devices in the nursery or home.

In one exemplary embodiment, the present subject disclosure is an infant soother. The infant soother includes a housing; a speaker; and a projector; wherein the projector is adapted to project a first light configuration onto a surface and transition to at least a second light configuration while the speaker generates an accompanying sound.

In another exemplary embodiment, the present subject disclosure is a smart nursery. The smart nursery includes an air modification device; a sensor that is separate from but wirelessly connected to the air modification device; and an electronic device which stores a software application that controls and communicates with the sensor and/or the air modification device.

In yet another exemplary embodiment, the present subject disclosure is a smart nursery. The smart nursery includes one or more devices adapted to communicate electronically; one or more sensors that are separate from but electronically connected to at least one of the devices; an electronic device storing an electronic application which communicates with at least one the devices or sensors; and a remote server system in communication with the electronic device and containing historical and operational information relating to the operation of the devices or the sensors.

### BRIEF DESCRIPTION OF THE DRAWINGS

Various exemplary embodiments of this disclosure will be described in detail, wherein like reference numerals refer to identical or similar components or steps, with reference to the following figures, wherein:

FIG. 1A is an exploded front perspective view of an infant soother, according to an exemplary embodiment of the present subject disclosure.

FIG. 1B is an exploded front perspective view of another infant soother, according to an exemplary embodiment of the present subject disclosure.

FIG. 1C is an exploded front perspective view of another infant soother, according to an exemplary embodiment of the present subject disclosure.

FIG. 1D is a front view of an infant soother and a monitor application for a smart device, according to an exemplary embodiment of the present subject disclosure.

FIG. 2A is a perspective view of an infant soother, according to an exemplary embodiment of the present subject disclosure.

FIG. 2B is a back view of an infant soother, according to an exemplary embodiment of the present subject disclosure.

FIG. 2C is a front view of an infant soother, according to an exemplary embodiment of the present subject disclosure.

FIG. 2D is a side view of an infant soother, according to an exemplary embodiment of the present subject disclosure.

FIG. 2E is a top view of an infant soother, according to an exemplary embodiment of the present subject disclosure.

FIG. 2F is a bottom view of an infant soother, according to an exemplary embodiment of the present subject disclosure.

FIG. 2G is a cross cut sectional view of an infant soother along Section A-A of FIG. 2C, according to an exemplary embodiment of the present subject disclosure.

FIG. 2H is a cross cut sectional view of an infant soother along Section B-B of FIG. 2D, according to an exemplary embodiment of the present subject disclosure.

FIG. 2I is a cross cut sectional view of an infant soother along Section A-A of FIG. 2C, according to an exemplary embodiment of the present subject disclosure.

FIG. 2J is a cross cut sectional perspective view of an infant soother along Section A-A of FIG. 2C, according to an exemplary embodiment of the present subject disclosure.

FIG. 3A is a front view of an infant soother, according to an exemplary embodiment of the present subject disclosure.

FIG. 3B is a cross cut sectional view of an infant soother along Section C-C of FIG. 3A, according to an exemplary embodiment of the present subject disclosure.

FIG. 3C is a side view of an infant soother, according to an exemplary embodiment of the present subject disclosure.

FIG. 3D is a cross cut sectional view of an infant soother along Section D-D of FIG. 3C, according to an exemplary embodiment of the present subject disclosure.

FIG. 3E is a top view of the infant soother.

FIG. 4A is a front view of an infant soother, according to an exemplary embodiment of the present subject disclosure.

FIG. 4B is a cross cut sectional view of an infant soother along Section E-E of FIG. 4A, according to an exemplary embodiment of the present subject disclosure.

FIG. 4C is a side view of an infant soother, according to an exemplary embodiment of the present subject disclosure.

FIG. 4D is a cross cut sectional view of an infant soother along Section F-F of FIG. 4C, according to an exemplary embodiment of the present subject disclosure.

FIG. 4E is a top view of the infant soother.

FIG. 5 is a schematic diagram on an infant soother and features, according to an exemplary embodiment of the present subject disclosure.

FIG. 6 is a schematic diagram of a sleep routine sequence, according to an exemplary embodiment of the present subject disclosure.

FIG. 7A is a schematic diagram of different light modes of an infant soother, according to an exemplary embodiment of the present subject disclosure.

FIG. 7B is a schematic diagram of light therapy, according to an exemplary embodiment of the present subject disclosure.

FIG. 8 is a schematic diagram of ages and stages for light therapy, according to an exemplary embodiment of the present subject disclosure.

FIG. 9 is a perspective view of a light projected from an infant soother onto a wall with a zoom and aperture features, according to an exemplary embodiment of the present subject disclosure.

FIGS. 10A, 10B, and 10C show several views of a light projected from an infant soother with a keystone correction feature, according to an exemplary embodiment of the present subject disclosure.

FIG. 11 is a schematic diagram of a sleep wake transition mode of an infant soother, according to an exemplary embodiment of the present subject disclosure.

FIG. 12A is a schematic diagram of an IoT ecosystem including an infant soother, according to an exemplary embodiment of the present subject disclosure.

FIG. 12B is a front view of various devices in an IoT ecosystem, according to an exemplary embodiment of the present subject disclosure.

FIG. 13A is a front view of a humidifier and a monitor application for a smart device, according to an exemplary embodiment of the present subject disclosure.

FIG. 13B is a front view of a UV-C humidifier and diffuser and a humidifier room meter sensor, according to an exemplary embodiment of the present subject disclosure.

FIG. 14A is a front view of an air purifier and a monitor application for a smart device, according to an exemplary embodiment of the present subject disclosure.

FIG. 14B is a front view of an air purifier and an air purifier room meter sensor, according to an exemplary embodiment of the present subject disclosure.

FIG. 15 is a front view of an air purifier and an air purifier room meter sensor, according to an exemplary embodiment of the present subject disclosure.

FIG. 16A is a front perspective view of a crib and various configurations of an air purifier sensor and a humidifier sensor, according to an exemplary embodiment of the present subject disclosure.

FIG. 16B is a front perspective view of a crib and a toddler bed with attached humidifier and air purifier sensors, according to an exemplary embodiment of the present subject disclosure.

FIG. 17 is a front schematic view of various humidifier and air purifier features, according to an exemplary embodiment of the present subject disclosure.

FIG. 18A is a front schematic view of various humidifier and air purifier features, according to an exemplary embodiment of the present subject disclosure.

FIG. 18B is a front schematic view of various humidifier and air purifier features, according to an exemplary embodiment of the present subject disclosure.

FIG. 19A is a front view of a nursery with a humidifier and an air purifier and related sensors in use, according to an exemplary embodiment of the present subject disclosure.

FIG. 19B is a front view of a nursery with a humidifier and smart application in use along with a monitor application on a phone, according to an exemplary embodiment of the present subject disclosure.

FIG. 19C is a front view of a nursery with a humidity sensor and an air sensor in use along with a monitor application on a phone, according to an exemplary embodiment of the present subject disclosure.

FIG. 19D is a front view of a nursery with a humidifier and an air purifier and related sensors in use along with a monitor application on a phone, according to an exemplary embodiment of the present subject disclosure.

FIG. 19E is a front perspective schematic view of an IoT system, according to an exemplary embodiment of the present subject disclosure.

FIG. 20 is a front perspective view of various monitor and sensor crib attachments, according to an exemplary embodiment of the present subject disclosure.

FIG. 21 is a diagram of an IoT ecosystem in communication with an IoT network, according to an exemplary embodiment of the present subject disclosure.

FIG. 22 is a diagram of an IoT ecosystem in a nursery, according to an exemplary embodiment of the present subject disclosure.

FIG. 23 is a schematic diagram of a smart device circuit, according to an exemplary embodiment of the present subject disclosure.

FIG. 24 is a schematic diagram of a remote server system, according to an exemplary embodiment of the present subject disclosure.

### DETAILED DESCRIPTION

The present subject disclosure describes infant or child soother devices that serve to create a circadian rhythm using light and/or sound. The devices use light of particular colors and intensities which are shown to assist in developing circadian sleep cycles. Further, sound is used to assist in the sleep therapy. The infant and child soother is connectable to a larger home smart system that monitors and controls a number of different variables in the location of a child, for example, a child's bedroom or nursery. It should be noted that the devices, systems, and methods described herein make frequent reference to infants, toddlers, or children, for sake of simplicity, but the present subject disclosure is not limited to children, but may also be useful and applicable to adolescents, adults, and even pets to establish sleep cycles, and monitor and control the conditions in a particular room. Particular embodiments of an infant soother will now be described in greater detail with reference to the figures.

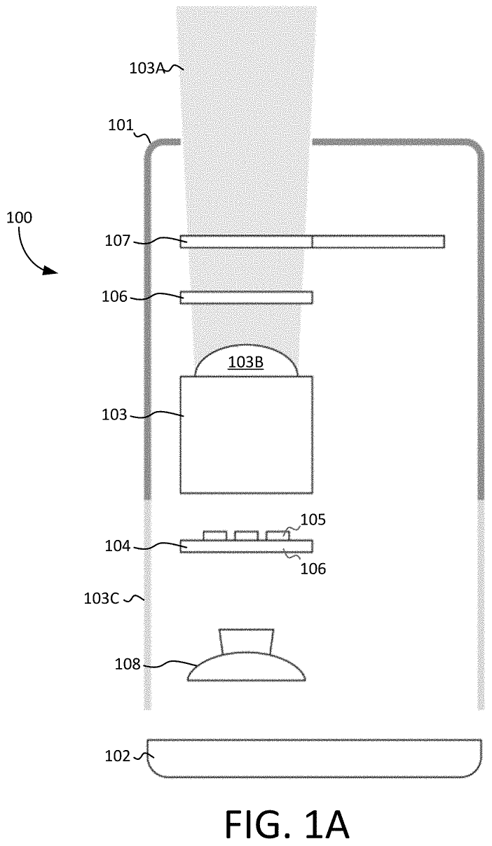

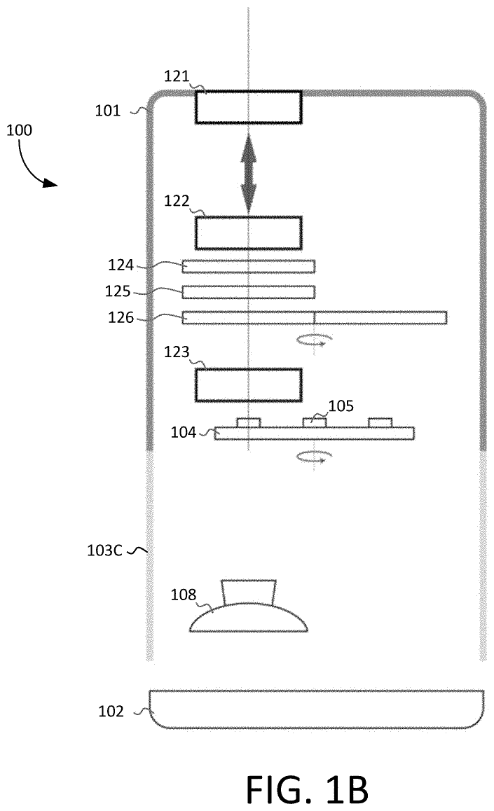

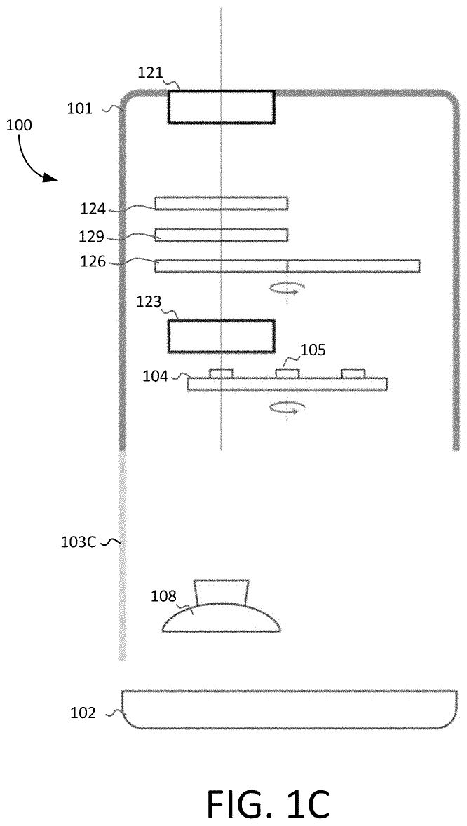

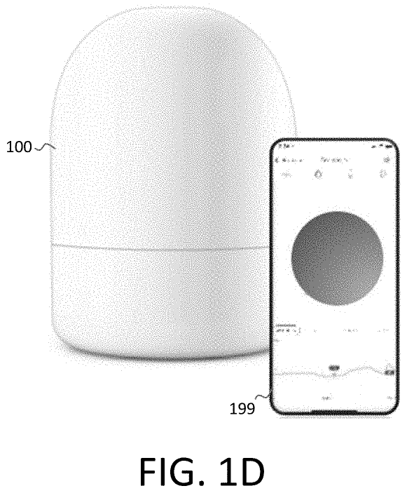

FIG. 1A illustrates an exploded front perspective view of an infant soother 100, according to an exemplary embodiment of the present subject disclosure. The infant soother 100 may include an outer housing 101 attached to a base 102. The outer housing 101 may be cylindrical, rectangular or any three-dimensional solid that may house the internal components of the infant soother 100 without departing from the broader aspects of the present subject disclosure. The outer housing 101 may be formed as a single piece or may include one or more sections that may be assembled to form the outer housing 101. The outer housing 101 may have an upper portion and a lower portion. A top housing may enclose an interior compartment formed by the outer housing 101 at the upper portion, while the base 102 may enclose the interior compartment formed by the outer housing 101 at the lower portion.

As shown in FIG. 1A, the infant soother 100 may include a projector 103 for projecting a light from the infant soother. The projector 103 is adapted to project a light 103A onto a surface, most likely a wall or ceiling in a room, that adopts the appearance of a sun, moon, star, sunset, sunrise and the like. These light projections are used to reinforce sleep cues and help parents sleep train their infants, toddlers and small children. The projector 103 and the projected light 103A may also be beneficial for older children and adults. The projector 103 may be housed in a projector housing that is disposed in the interior compartment. The projector 103 may include at least one light source 104. The light source 104 may be a light-emitting diode (LED) light or a light bulb. The light source 104 may include more than one colored lights 105. As shown in FIG. 1A, the LED light source 104 includes three different colored lights 105, including a white light LED, an orange light LED and a yellow light LED that are attached to an LED circuit board 106. Additional colored lights 105 that generate light according to different wavelengths of the electromagnetic spectrum may be included in the light source 104, including any colored light from the visible light spectrum, ultraviolet (UV) light spectrum and Infrared (IR) spectrum. The light source 104 may be adapted to generate light at various intensities and frequencies. The light source 104 may be adapted to generate ambient light 103C in addition to lighting 103A up the projector 103 and/or at least one additional ambient light may be included in the infant soother.

The projector 103 may include at least one lens 103B in the projector 103 housing for focusing the light 103A generated by the light source 104. A plano-convex lens may be disposed closest to the light source 104 for focusing, collecting and collimating the rays of light to a focused point. At least one additional lens may be included in projector 103 housing for focusing the image generated by the projector 103 onto a wall or surface. The lenses 1038 may be simple or compound. The lenses 103B may be spherical with either surface comprising a concave or convex surface. The lenses 103B may have at least one axis. The optical surfaces of the lenses 103B may be biconvex, plano-concave, biconcave, or have a positive meniscus or a negative meniscus. The lenses 103B may include at least one filter and may incorporate one or more dichroic materials.

The projector 103 may include a zoom feature 106. The zoom feature 106 may include an aperture and/or a zoom lens. The zoom lens may be a parfocal lens. The projector 103 may include at least one prism, mirror or reflective surface. The projector 103 may include a slide holder for projecting an image onto a surface. The projector 103 may include an eclipse feature 107, such as an aperture, screen or opaque panel for blocking the projector light 103A in order to model a sunrise, sunset, or lunar cycle. The eclipse feature 107 rotates in time to project different light patterns, as will be explained in detail below. A speaker/microphone 108 further provides selected sounds and listening ability. These features will be explained in detail below. FIG. 1B shows an alternate configuration of the infant soother 100 having a top lens 121, middle lens 122 and a bottom lens 123 for projecting the light on an external surface. The top lens 121 may be adapted to move side to side, tilt or otherwise suitably realign a distorted image projected onto an angled surface in order to generate a keystone correction effect that focuses and controls the projected light. The keystone feature may correct the shape of the projected light and create clear and defined edges of the projected light. The middle lens 122 may be adapted to move closer or farther away from the top lens 121 in order to control the zoom feature and the focus of the projected image. Dichroic film 124 and masking film 125 may be disposed between the middle lens 122 and the bottom lens 123 in order to modulate the projected light. The masking film 125 may be adapted to generate a halo effect for the projected light. An eclipse shade 126 may also be disposed between the middle lens 122 and bottom lens 123 in order to create the eclipse feature of the projected light. The eclipse shade 126 may be adapted to rotate or move in relationship to the LED board 104 and the LED lights 105 disposed therein. The eclipse shade 126 and/or the LED board 104 or LED lights 105 may be adapted to move within the soother housing 101, which is adapted to connect with base 102. The relationship between the movement of the LED lights 105 and eclipse shade 126 and dichroic film 124 generate the modeled light sequence, sunrise, sunset, or lunar cycle. A speaker/microphone 108 is disposed within the housing 101 and will be described in more detail below.

FIG. 1C is an alternate configuration of the infant soother 100 having a top lens 121 and a bottom lens 123. However, in this embodiment, an aperture plate 129 replaces the middle lens 122 (of FIG. 1B) in order to zoom and focus the projected light. All of the embodiments of the infant soother may be adapted to dissipate heat through venting or other means. The other components contained in this soother configuration operate substantially as discussed in the other embodiments disclosed above.

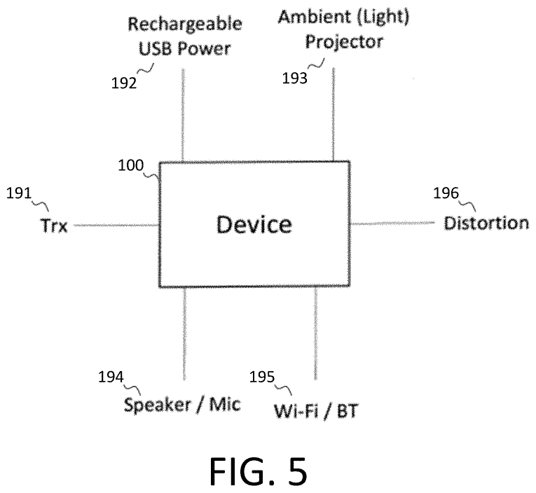

As shown in FIGS. 1A-1C and 5, the infant soother 100 may include at least one speaker and/or at least one microphone (shown combined as 108). The infant soother 100 may include a processor, a controller, an integrated CPU, printed circuit board, at least one circuit, a power supply, at least one USB port, a timer and memory for storing programs, routines and data. The infant soother 100 may include a communication array that may include a transmitter, transducer, transceiver, an antenna, and may be adapted for communication using any of a variety of custom or standard wired (Ethernet, LAN, HomePlug, etc.) or wireless protocols, such as: IEEE 802.15.4, Wi-Fi, ZigBee, 6LoWPAN, Thread, Z-Wave, radio, Bluetooth Smart, WirelessHART, MiWi and/or including any other communication protocols. These features are not shown for sake of simplicity but are fully appreciated by one having ordinary skill in the art after consideration of the present subject disclosure.

The infant soother 100 may include at least one sensor. The sensor may be a humidity sensor, a temperature sensor, an acoustic sensor, a sound sensor, a video sensor, a vibration sensor, a chemical sensor, an electric current sensor, an electric potential sensor, a magnetic sensor, a radio sensor, a radiation sensor, a navigational sensor, a position sensor, an angle sensor, a displacement sensor, a distance sensor, a speed sensor, an acceleration sensor, an optical sensor, a light sensor, an imaging sensor, a photon sensor, a pressure sensor, a force sensor, a density sensor, a level sensor, a thermal sensor, a heat sensor, a proximity sensor, a presence sensor, a sonar sensor, a micro-electrical mechanical system sensor, a radar sensor, an ultrasonic sensor, an air pollution sensor, an air quality sensor, a carbon monoxide sensor, volatile organic compound (VOC) sensor, a biometric sensor and/or any combination of the above listed sensors. These features are not shown for sake of simplicity but are fully appreciated by one having ordinary skill in the art after consideration of the present subject disclosure.

The infant soother 100 may include a display that may be a touchscreen, a clock with an alarm, timer, physical buttons, a child lock and may be adapted to receive voice commands through the microphone. The microphone 108 may be adapted to capture noise such as infant crying from the environment. The speaker 108 may be adapted to play music, white noise, pink noise and the like, for soothing purposes.

As shown in FIG. 1D, the infant soother 100 may be adapted for use with a monitor application (app) for smart devices 199. A user may input commands into the monitor app that will then be received by the infant soother 100. The monitor app may receive updates, alerts and information transmitted from the infant soother, a remote server, a cloud computing network, another client or endpoint device and/or any other communication architecture. The monitor app may communicate directly with the infant soother 100 and/or the sensors, and/or may communicate with the infant soother through a network and/or cloud server.

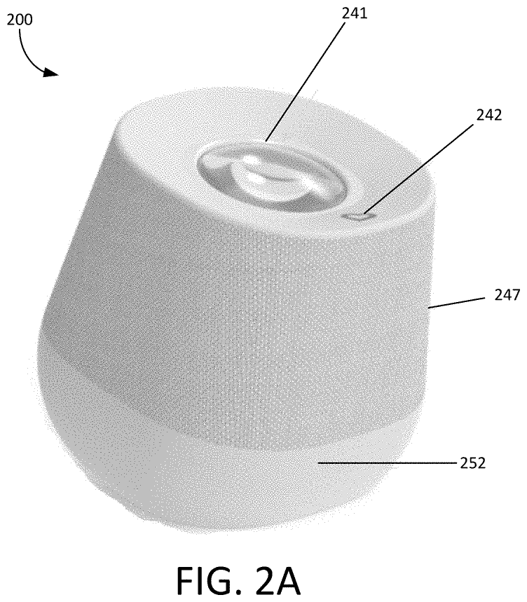

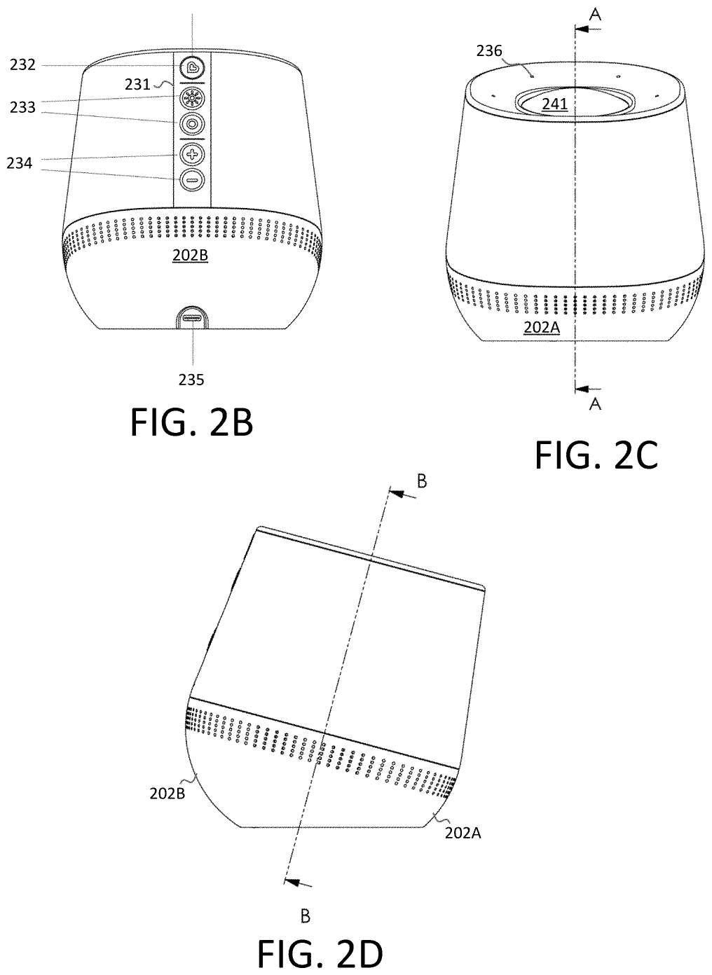

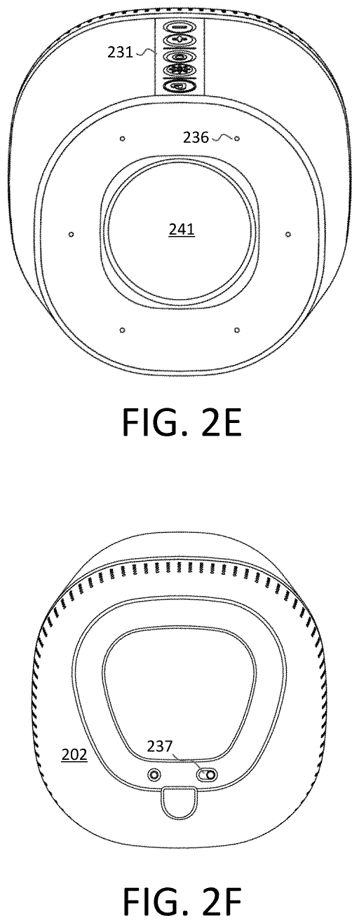

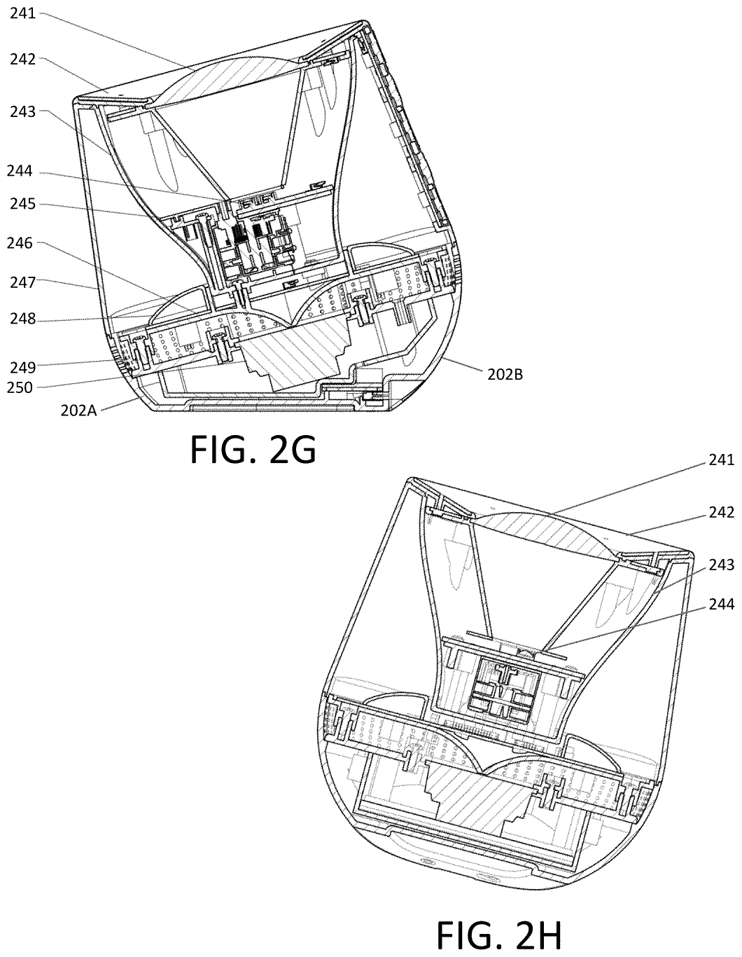

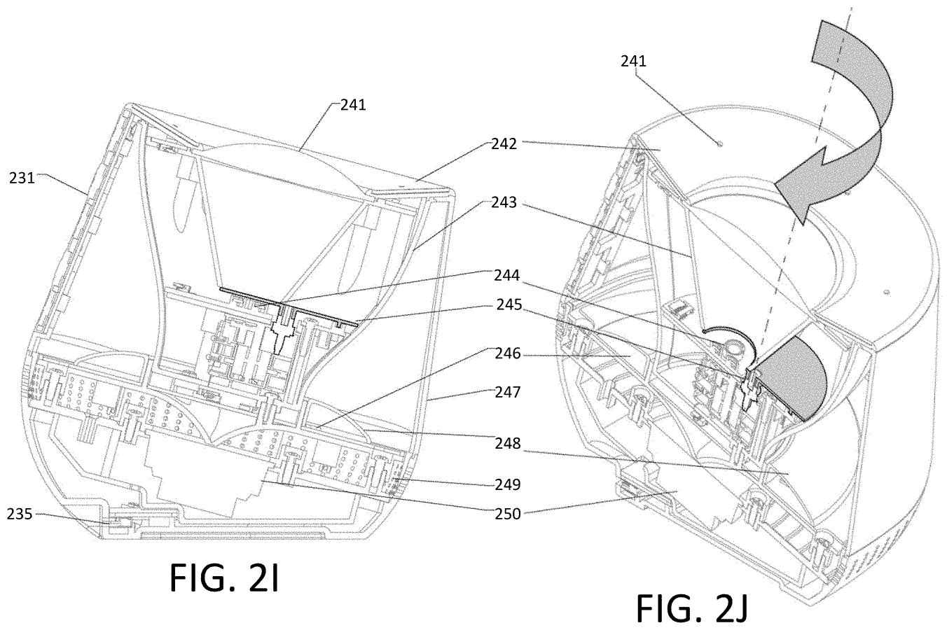

FIG. 2A-2J show an infant soother 200, according to an exemplary embodiment of the present subject disclosure. FIG. 2A shows a front perspective view of the exemplary infant soother 200, showing the projected light 241, ambient light 247, one touch operation 242, and a clock 252.

FIGS. 2B, 2C, 2D, 2E, and 2F show back, front, left side (right side would be symmetrically opposite), top, and bottom views, respectively, of the infant soother 200. As shown in FIG. 2B, a keypad control panel 231 includes a number of buttons to alter the output of the soother 200. Exemplary control buttons include, but are not limited to, favorite button 232. Ambient light increase/decease buttons 233, and volume increase/decrease buttons 234. A port 235 allows power input to the soother device 200. The port may be, for example, a USB-C port, or other port to provide power.

As shown in FIGS. 2C, 2D, and 2E, the soother is asymmetrical so that the base 202 has a front portion 202A which is smaller than a back portion 202B. This asymmetry provides a forward tilt to the soother 200 that allows the soother to project light on a ceiling or wall that is not directly overhead of the soother 200. Stated differently, the asymmetrical design allows the projection of light on a ceiling or wall away from the soother 200 so that the soother has greater flexibility of light projection across the ceiling/walls of a room when the soother is placed on furniture, such as a night stand.

As shown in FIGS. 2C and 2E, one or more holes 236 allows access to a microphone which is used to detect crying or other sounds from the room in which the soother 200 is positioned. FIG. 2F shows one or more switches 237 positioned on the base 202 of the soother 200 to allow the unit to be turned on/off or other suitable control.

FIG. 2G shows cross sectional diagram across Section A-A shown in FIG. 2C. A lens 241 is positioned atop the device 200, surrounded by a one touch surface 242. The one touch surface 242 may be programmed to control a number of features in the soother 200, including but not limited to all of the features presented as buttons 232, 233, 234 in the control panel 231. The one touch surface 242 may be programmed to control other features as well. Funnel 243 contains the projection light from bleeding out. One or more projection LEDs 244 are positioned under the lens 241. Disk 245 rotates via motor thereby projecting various light options out of the soother device 200. Ambient light LED 246 may be white or RGB. External wall outer diffuser 247 and inner wall inner diffuser 248 allow a soft outward projection of ambient light produced by the ambient light LED 246. One or more speaker holes 249 surround the outer circumference of the soother device 200. A speaker 250 is positioned internal to the soother device 200 and used to project sounds or voices out of the device 200.

FIG. 2H shows cross sectional diagram across Section B-B shown in FIG. 2D. A lens 241 is positioned atop the device 200, surrounded by a one touch surface 242. Funnel 243 contains the projection light from bleeding out. One or more projection LEDs 244 are positioned under the lens 241.

FIG. 2I shows the inverse cross sectional diagram across Section A-A shown in FIG. 2C. FIG. 2J shows a perspective view of FIG. 2I. The various features shown and described in FIGS. 2A-2H are shown in more detail.

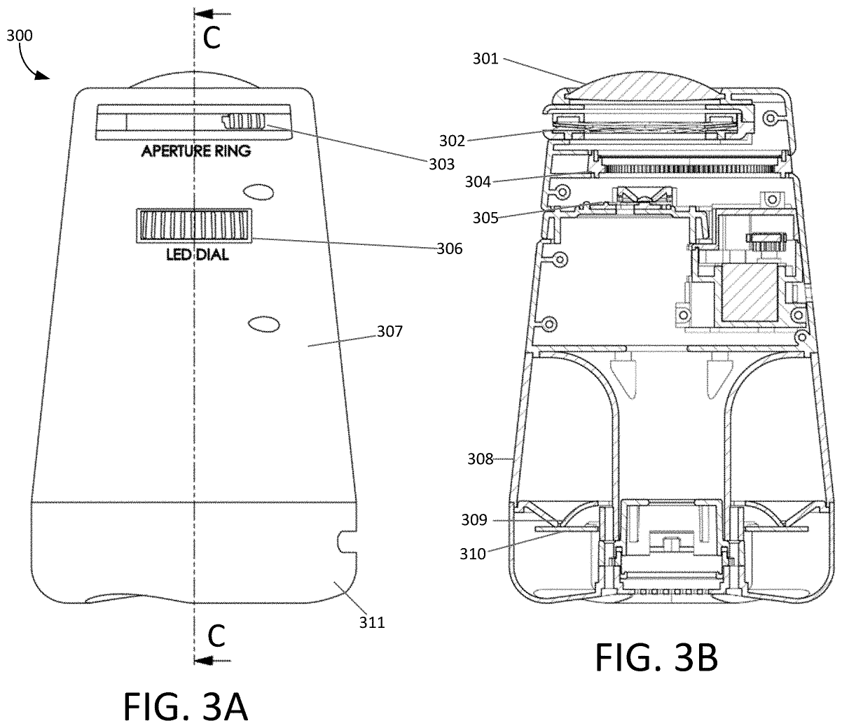

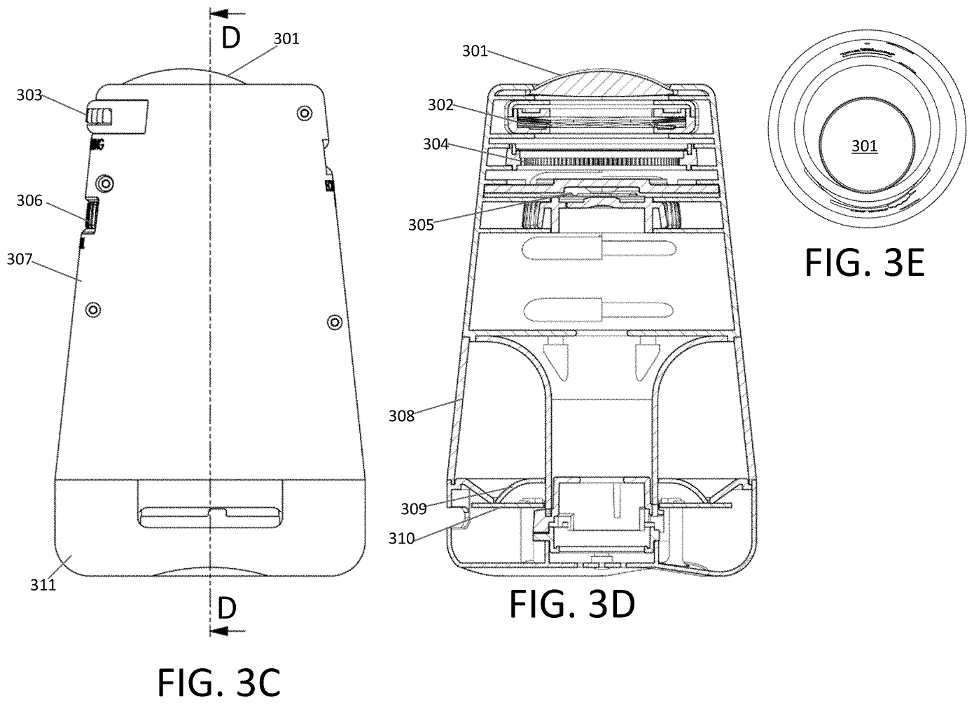

FIG. 3A shows a front view of another exemplary embodiment of an infant soother 300 according to the present subject disclosure. FIG. 3B shows a cross sectional diagram across Section C-C shown in FIG. 3A. FIG. 3C shows a side view, and FIG. 3D shows a cross sectional diagram across Section D-D shown in FIG. 3C. In FIG. 3E, a lens 301 is positioned atop the device 300. Zoom feature 302 allows control of light display through rotation dial 303. The eclipse feature 304 rotates to provide different light features, as will be described in more detail below. An LED dial 306 controls the intensity of the projected light. Top housing 307 mates with base 311 and contains the features of the device 300 and blocks out the light produced in the interior. An ambient light diffuser 308 allows a soft outflow of light as will be described in detail below. Ambient light inner diffuser 309 projects light produced by the ambient light LED 310.

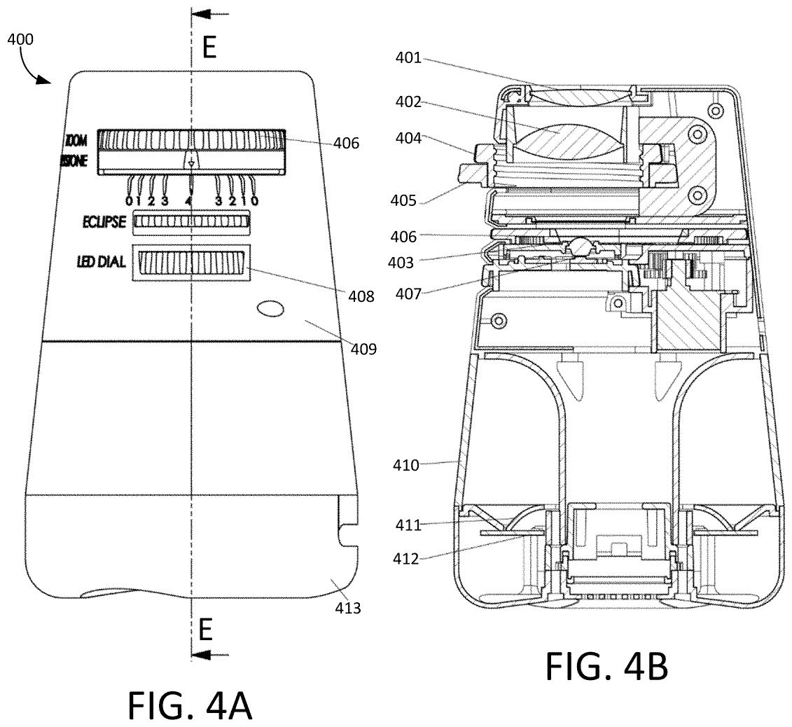

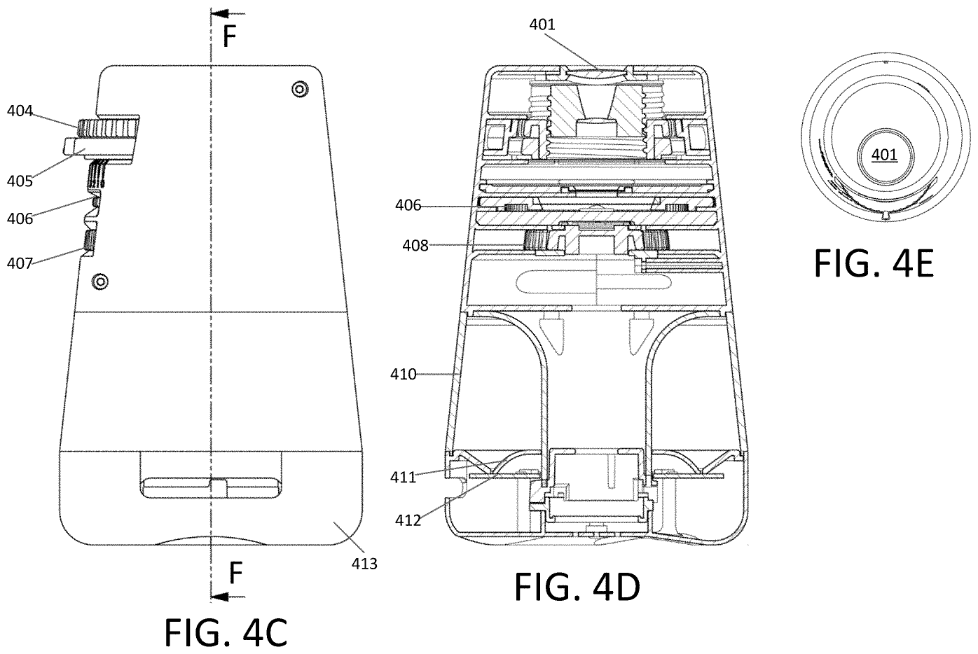

FIG. 4A shows a front view of another exemplary embodiment of an infant soother 400 according to the present subject disclosure. FIG. 4B shows a cross sectional diagram across Section E-E shown in FIG. 4k FIG. 4C shows a side view, and FIG. 3D shows a cross sectional diagram across Section D-D shown in FIG. 3C. In FIG. 4E, a lens 401 is positioned atop the soother device 400. Further, a middle lens 402 and a bottom lens 403 are positioned on the same central axis of the top lens 401. Zoom feature 404 is controllable through up and down and rotation motions through turning dial 406. Further, keystone feature 405 is also controllable through a turning dial. Eclipse feature 406 is positioned above the LED 407, which can be controlled through a rotating dial 408. The LEDs may be various colors including, for example, white, orange, yellow, etc. Top housing 408 mates with base 413 and contains lights produced therein. Ambient light outer diffuser 410 and ambient light inner diffuser 411 serve to project light produced by the ambient light LED 412.

As shown in the simplistic diagram of FIG. 5, infant soother 100 may have input transceiver 191. Further components include a speaker/microphone 194 and WiFi/Bluetooth 195 capability. A rechargeable USB power source 192 powers the device 100. An ambient light projector 193 provides one optional light source. The distorted light 196 produces another optional light source.

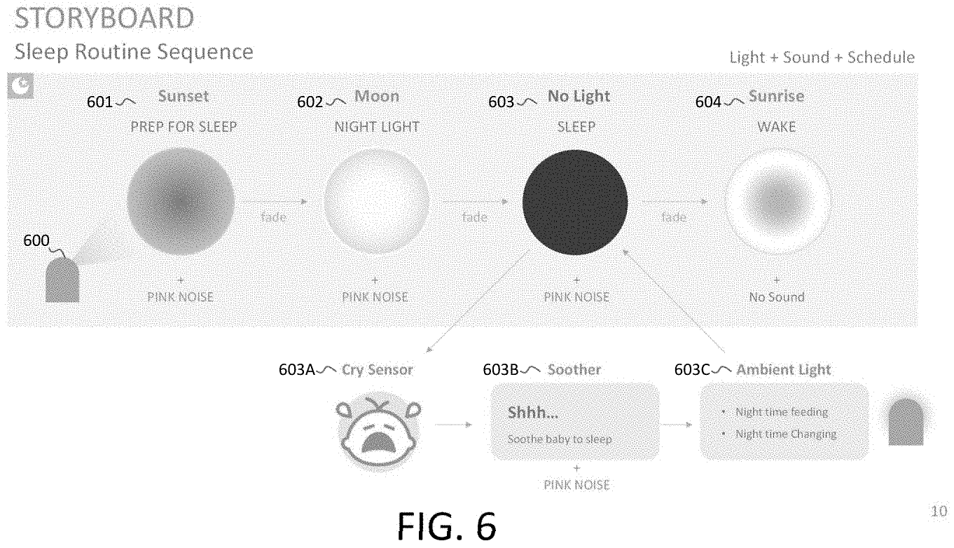

FIG. 6 is a schematic view of a sleep routine sequence. The sleep routine sequence may assist with the sleep training of infants, toddlers and young children. Toddlers and young children thrive on routine and knowing what to expect and what comes next helps them to feel confident and in control. The sleep training also functions on a subconscious and physiological level that promotes good health and sleep habits for infants, toddlers and young children. An exemplary sleep routine or method of sleep training may begin with the infant soother 600 projecting a source of light onto a wall, ceiling or surface. The infant soother may also play an accompanying sound that matches the projected light. The sound may be white noise, static, pink noise, balanced tones, even tones, relaxing tones, sleep tones, customized recordings, custom playlists or streams from apps such as Spotify, Amazon music, and Apple music, podcasts, stories, and/or sing song scripts.

As shown in FIG. 3, the projected light is a sunset 601 that may be paired with pink noise. The user may choose different sound and light profiles that are adapted to their needs. The sunset may be an orange, red or yellow color and may match the lighting characteristics (3000-4000 Kelvin) of an actual sunset. The sunset may last for a given period of time that is either preset or chosen by the user. The sunset 601 allows the user and child to prepare for sleep and engage in their usual sleep routine, ritual and/or procedure.

After the sunset period 601, the infant soother may project a new light or image that corresponds to a moon period 602. The infant soother may fade from the sunset 601 to the moon light 602 or transition between the two settings through a transition pattern or sequence. The moon light 602 may be a night light with a warm, white glow. The moon light may also be paired with a given sound profile, such as pink or white noise. The moon light period 602 may exhibit approximately the same color temperature as the sunset (around 4000 K) or may adopt a different color temperature chosen by the user.

The moon period 602 may then transition to a sleep period 603. The sleep period 603 may not include any projected light, but may play a given sound profile, such as pink noise, white noise, ambient music, and the like. During the sleep period 603, the infant soother 600 may be monitoring the environment for cues that the infant is awake. For example, the microphone may detect noise in the nursery. The infant soother 600 may compare the sound profile of the noise detected to an internal database or a database of sounds stored on a remote server that is connected to the infant soother 600 or monitor app through a cloud computing network. If the sound detected meets the profile of a stored sound profile that corresponds to a state such as crying 603A, the infant soother 600 may activate a soothing noise 603B to lull the infant back to sleep. The infant soother may active a light or light sequence 603C to calm the infant. If the intensity of the crying increases beyond a certain preset tolerance or given threshold, or the crying persists past a certain time limit, the infant soother 600 may alert the caregiver to the infant's ongoing discomfort. During the sleep period the infant soother 600 may be adapted to activate an ambient light 603C for night-time feeding or changing. The ambient light may be controlled by the monitor app, physical button on the soother, and/or may be voice activated or motion activated by a motion or gesture sensor.

In the morning, the sleep period may transition to a sunrise period 604 characterized by a brighter color temperature (above 4000K). The sunrise period 604 may include a paired sound profile or may not include any sound. The sunrise period 604 may transition from a sliver of light to a full sun-like appearance. The projected light may be photo-realistic, abstract, or cartoonish in tone and appearance. The brighter sunrise period helps to train the infant and the infant's brain that it is time to wake up and may help to establish a healthy routine or sleep habit. The infant soother may also include a calming blue light setting for naps during the day that may be paired with a given sound profile such as soothing noises.

The infant soother 600 and the sleep routine features incorporated therein may be controlled remotely by the monitoring app or by a remote control. The infant soother 600 and monitoring app may be adapted to educate consumers on best sleep practices and how to best use the infant soother and sleep routine system. The monitoring app may provide informative information related to the baby's age and important milestones, such as teething, feeding, sleep training, potty training, etc. The infant soother 600 and monitoring app may provide suggestions and guidance to the user based upon gathered data and user history. The soother 600 and monitoring app may include a scheduling feature and the ability to create and optimize a bedtime routine and program.

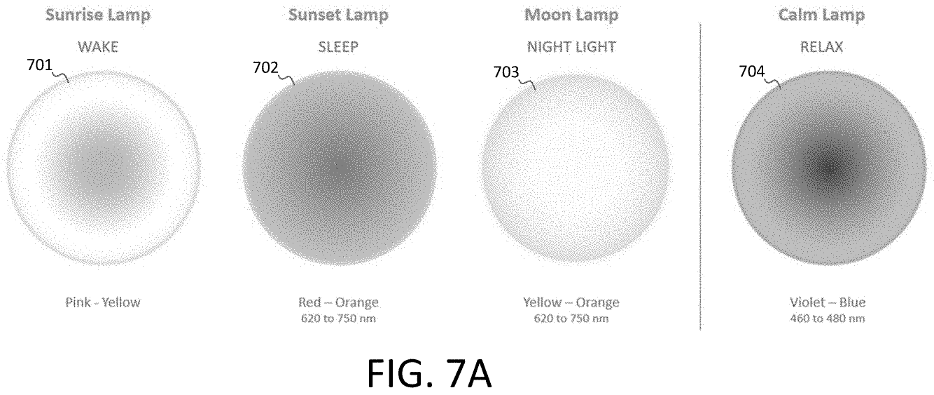

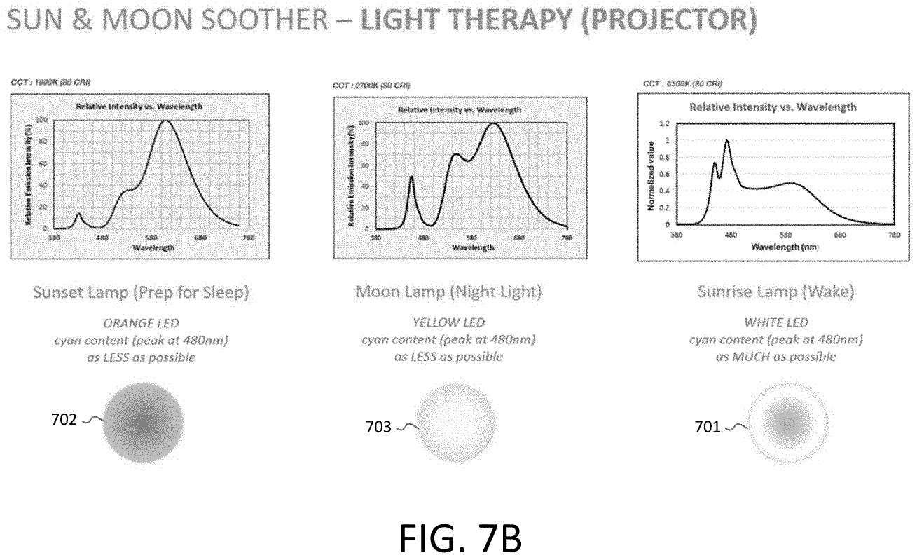

FIG. 7A shows further details of the projected light settings and the infant soothers light therapy functions. The infant soother may be adapted for bright light therapy. The projected light may generate a light that may be adjusted between 2,500 and 10,000 lux for optimizing the circadian rhythms and sleep cycles of an infant, child or user. The projected light may be adapted to filter certain wavelengths of the electromagnetic spectrum, such as blue, green and white light, in order to protect and maximize melatonin production and healthy sleep. The sunset lamp setting may generate light that is pink-yellow 701 which is suitable during for example sunrise or the wake hours. The sunset lamp setting may generate light that is red to orange 702 and may be characterized by light with wavelengths between 620-750 nm that has been proven to support the highest levels of melatonin production. Warm light in the red-orange 702 spectrum is recommended by sleep therapists, physiologists and NASA scientists as the optimum color for soothing sleep. The moon lamp setting may also generate light in the 620-750 nm range with a more yellow-orange color profile 703. The calm light setting may generate violet-blue light 704 around the 460-480 nm wavelength spectrum in order to generate a soothing effect. Blue light therapy has been shown to help children relax and may be used through the day to calm infants and help them transition out of a fussy state or fit. FIG. 7B shows an exemplary light therapy projector using sunset lamp 702, moon lamp 703, and sunrise lamp 701.

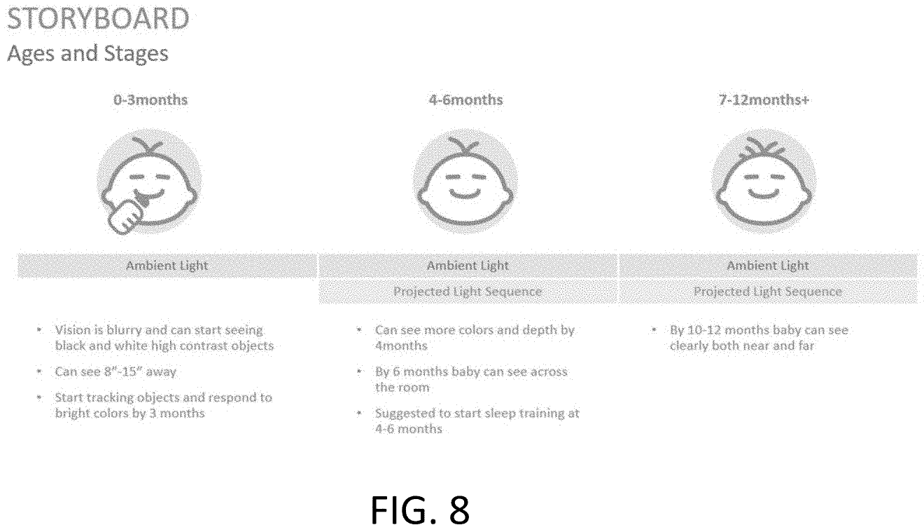

FIG. 8 shows that the recommended exposure to light varies between ages. For infants of 0-3 months, only ambient light is recommended. This is because the vision is blurry and they can start seeing black and white high contrast objects. They can see 8″-15″ away, and may start tracking objects and respond to bright colors by 3 months of age. For 4-6 month old infants, vision becomes more clear. They can see more colors and depth by 4 months, and by 6 months the infant can see across the room. This is the time period when sleep training start is suggested. In the 7-12+ months age group, sleep training is further recommended. By 10-12 months, the baby can see clearly both near and far.

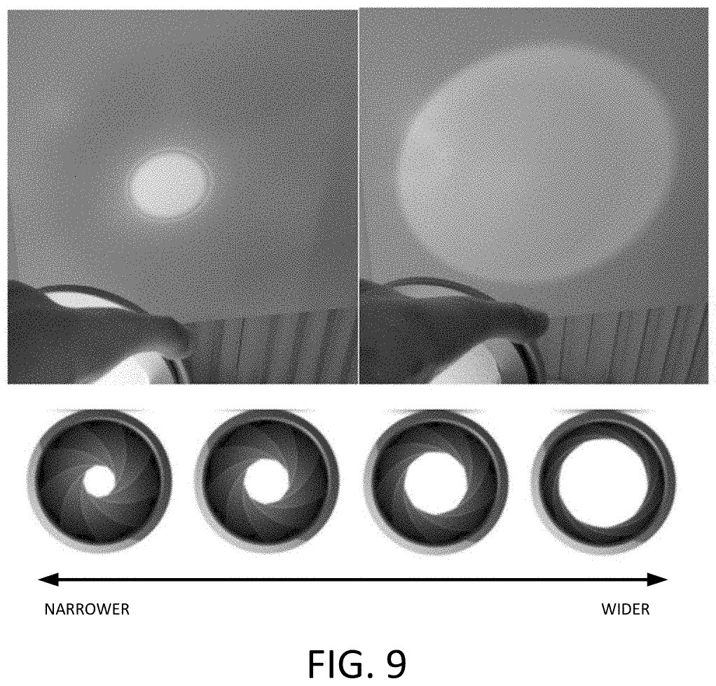

FIG. 9 is an image of the sunset projected onto a wall and various aperture settings that may be used to change the shape and size of the projected light, with a narrower aperture setting producing a smaller and sharper image, and a wider aperture setting producing a larger less sharp image. A zoom function may be incorporated into the projector for adjusting the light for different distances, surface configurations and shape, diameter and size parameters.

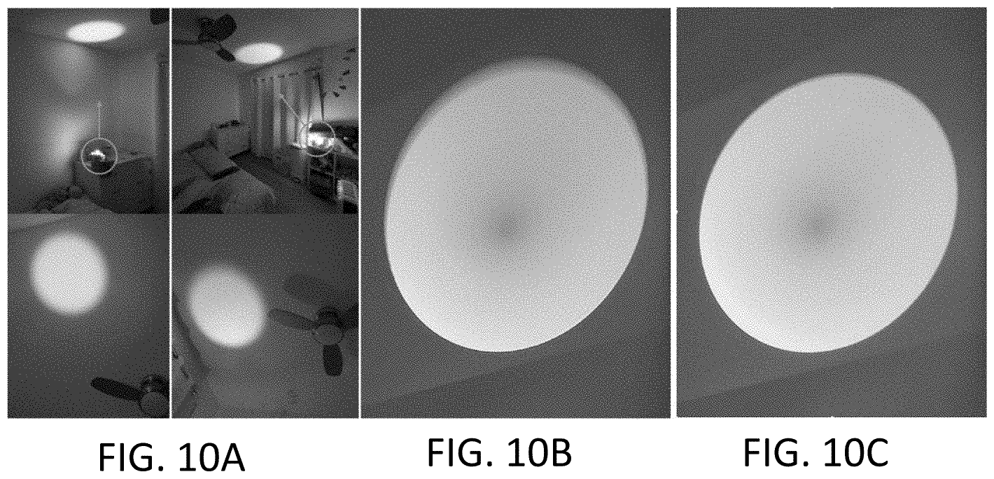

FIGS. 10A, 10B, and 10C show various images demonstrating the keystone effect of an uncorrected projected light. The keystone effect is the apparent distortion of an image caused by projecting it onto an angled surface. Generally, the keystone effect causes a projected image to be larger at the top than the bottom of the image and some areas of the image may be out of focus. FIG. 10A shows various examples of the keystone effect distortion of the produced image based on location of the image produced with a more focused bottom side and a wider and more disturbed top side when the image is projected on a surface at an angle. FIG. 10B shows a close-up image of the keystone effect showing that the top side of the image appears larger because the light is more disturbed while the bottom side of the image appears narrower and more focused. The infant soother may include a keystone correction feature in order to cancel out or minimize the keystone effect and other distortions. FIG. 10C shows a corrected image with the top and bottom of the image being more equally sharp and even sided. This produces a more pleasing and less distracting image to the infant.

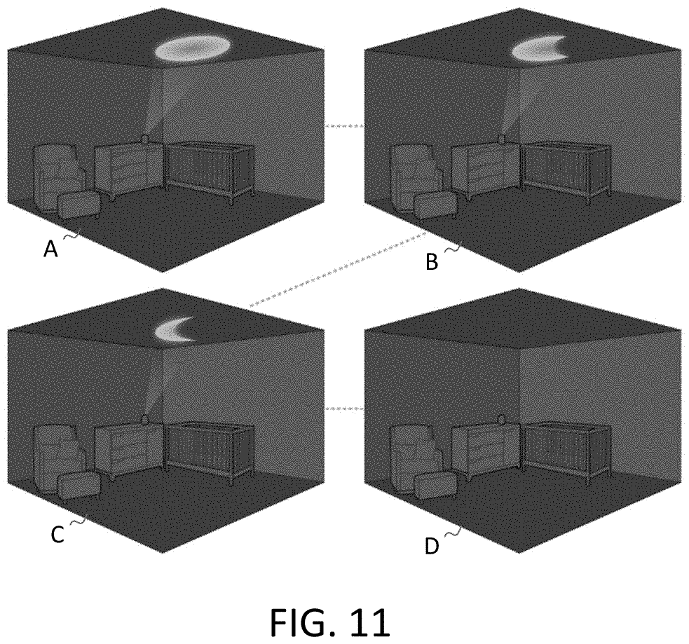

FIG. 11 shows a sleep wake transition between the different sleep periods that may be incorporated into the sleep routine or method of sleep training. The eclipse feature of the projector may block or filter a portion of the light generated by the light source in order to transition between the lamp settings of each period. As shown in FIG. 11, the moon slowly transitions from a full-moon (designated as A), to a half-moon (designated as B) to a crescent moon (designated as C) and then no light (designated as D) for the sleep period. The reverse procedure (D to C to B to A) may be effected in order to simulate a sunrise, or the eclipse feature may block the light source in a different fashion to convincingly mimic a sunrise or sunset. The eclipse feature may be utilized to generate a projected light that corresponds to the lunar cycle, or different depictions of the stars, solar system, constellations, or the like.

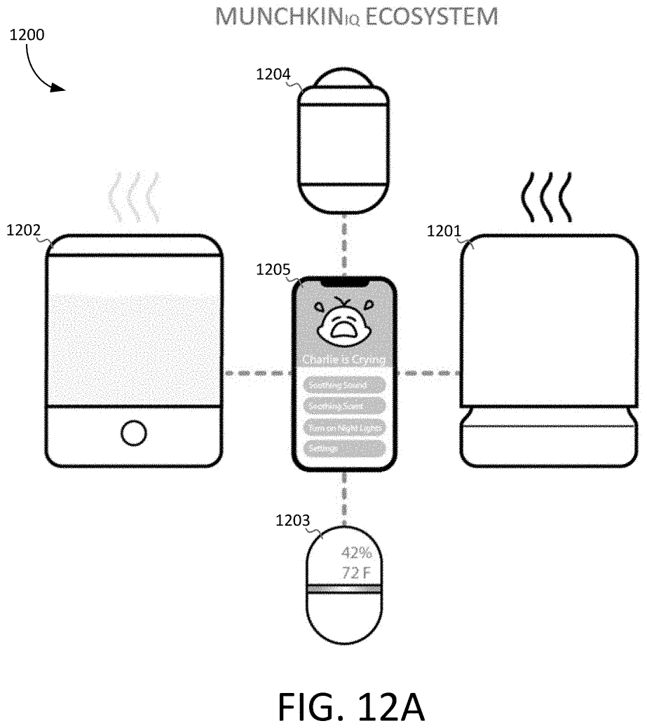

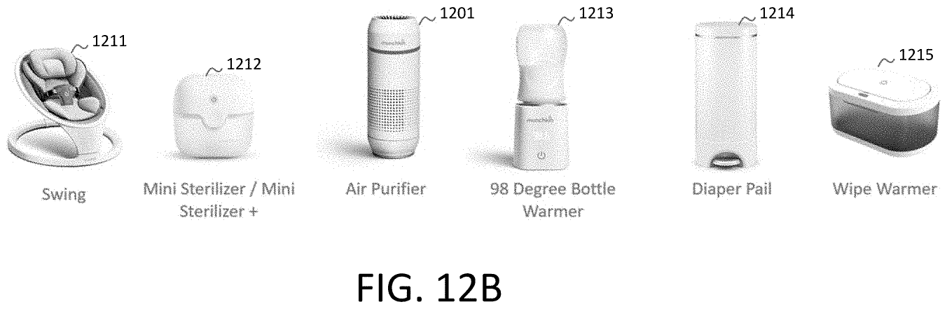

FIG. 12A is a schematic diagram of several products in an IoT ecosystem 1200 designed and adapted for a nursery and caregivers and children. As shown in FIG. 12, the IoT ecosystem 1200 includes an air purifier 1201, a humidifier 1202, at least one sensor 1203, and the infant soother 1204. The IoT ecosystem 1200 may be part of a local network, a client-server network, or cloud computing network. The smart devices 1205 in the IoT ecosystem 1200 are connected to the monitoring app that may be accessed and controlled from a compatible smart device such as a phone or computer. The IoT ecosystem 1200 may be compatible with other smart hub devices and may be integrated into existing home management systems. FIG. 12B shows various smart products that may be included in the IoT ecosystem 1200 such as, but not limited to, a swing 1211, sterilizer 1212, bottle warmer 1213, diaper pail 1214 and wipe warmer 1215. The IoT ecosystem 1200 may incorporate other smart devices and/or Bluetooth products such as speakers, headphones, sound bars, smart hubs and the like.

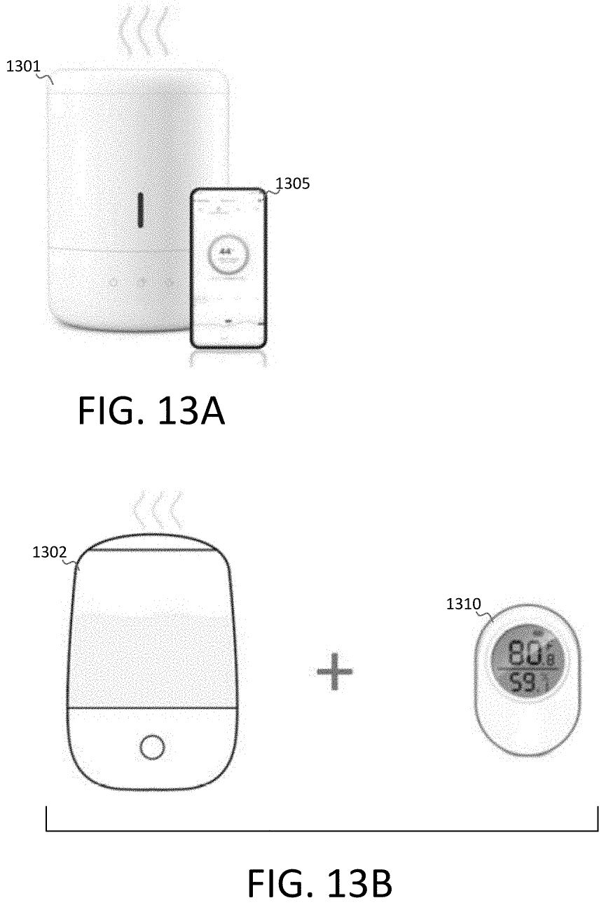

FIG. 13A is front view of a humidifier 1301 and an accompanying monitor application for a smart device 1305. The humidifier 1301 may be adapted to control or adjust the humidity in a room or environment by creating water vapor and expelling it into the air. The humidifier 1301 may be an evaporative humidifier having a reservoir, wick and a fan. The humidifier 1301 may be a vaporizer that heats or boils water in order to release steam or moisture into the air. The humidifier may be an ultrasonic humidifier.

FIG. 13B shows a humidifier and diffuser 1302 that incorporates UV-C light in order to sterilize the water before releasing it into the environment. The humidifier 1302 may include at least one humidifier room meter sensor 1310 in communication with the humidifier 1302 and the monitoring app 1305 that detects ambient environmental characteristics such as humidity and temperature.

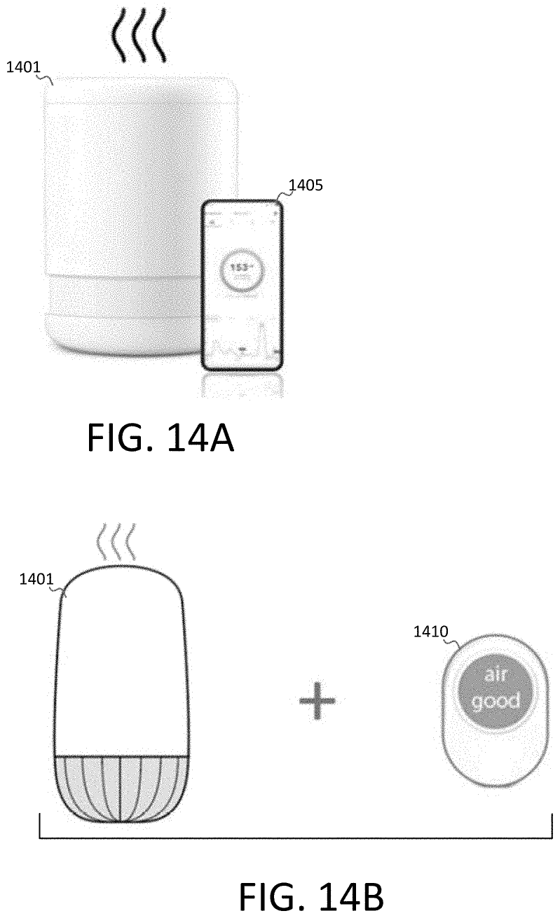

FIG. 14A shows an air purifier 1401 and an accompanying monitor application for a smart device 1405. The air purifier 1401 may be adapted to remove contaminants from the air in order to improve the indoor air quality. The air purifier 1401 may incorporate active or passive air purifying technologies. Active air purification releases negatively charged ions into the air in order to cause pollutants to stick to surfaces. Passive air purification involves filtration. The air purifier 1401 may use high-efficiency particulate air (HEPA) filters, activate carbon filters, ionizers, ultraviolet germicidal irradiation, photocatalytic oxidation, ozone generators, ROS purifiers, and other purification technology to purify the air in a room. The air purifier 1401 may be adapted for use with custom filters designed to filter different environments. The air purifier 1401 may be in communication with and controlled by the monitor app 1405. As shown in FIG. 14B, the air purifier 1401 may include at least one room meter sensor 1410 in communication with the air purifier 1401 and monitoring app 1405 for detecting the air quality and characteristics of the environment.

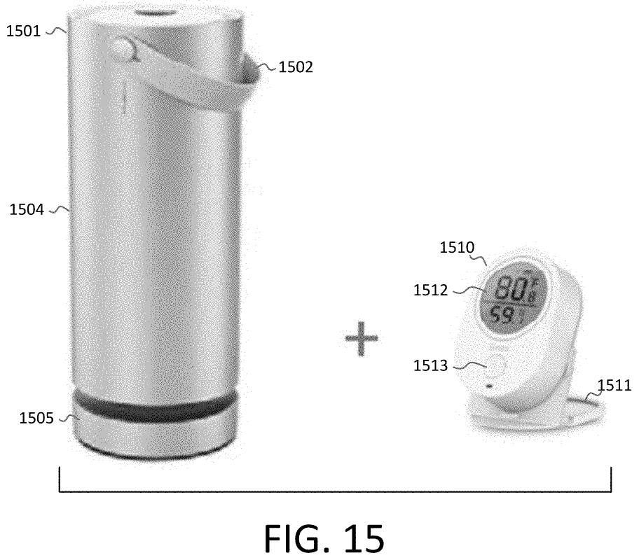

FIG. 15 shows an embodiment of the air purifier 1501 and air purifier room meter sensor 1510. The air purifier 1501 may be portable and may include a carrying strap 1502. The air purifier may have a brushed metal housing 1504 and may include a base 1505 that may be detachable. The sensor 1510 may include a base 1511 that also functions as a clip. The senor 1510 may have a display 1512 and a user control interface 1513 for controlling the sensor 1510.

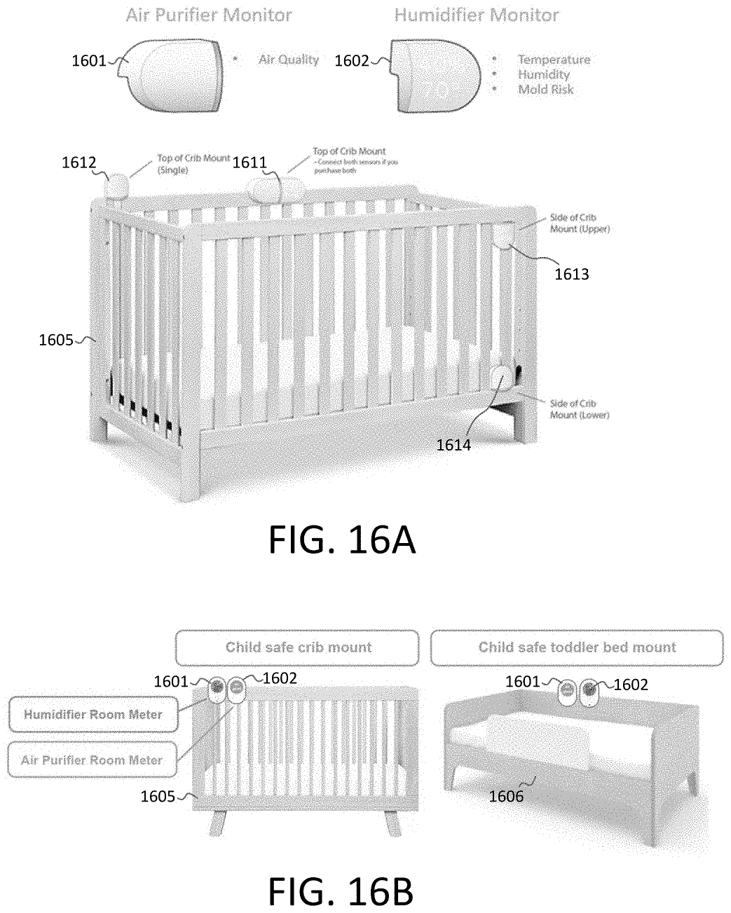

FIGS. 16A-16B show an air quality sensor 1601 and a humidity sensor 1602 that may be modularly attached together and/or affixed to a crib 1605, bed 1606 or other surface. The modular sensors 1601/1602 may be adapted to mate with other sensors, or with their respective home device. That is, the humidity sensor 1602 may be adapted to mate with the humidifier, while the air purifier sensor 1601 may be adapted to mate with the air purifier. The sensors 1601/1602 may be adapted to attach together 1611 to the top of the crib 1605, or attach separately 1612 to the top of the crib 1605, or between the bars of the crib 1605, at the upper mount 1613 or lower mount 1614 position. In FIG. 16B, sensors 1601/1602 are shown being mounted separately at the top portion of the crib 1605 and bed 1606. The air sensor 1601 may detect the quality of the air and/or the presence of carbon monoxide. The humidity sensor 1602 may detect the ambient temperature, humidity and the risk of mold formation. The sensors 1601/1602 may be adapted to mate with more than one sensor. Each sensor 1601/1602 may include a display. When combined, the modularly attached sensors 1601/1602 may be adapted to generate a single display.

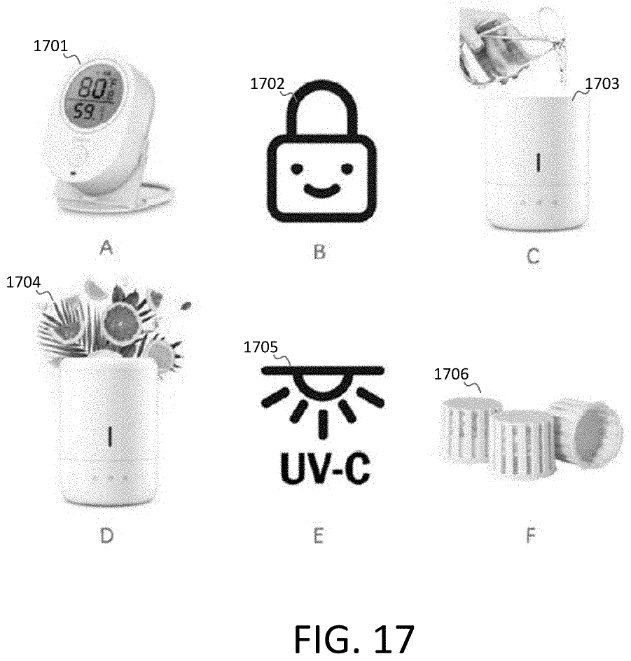

FIG. 17 is a schematic drawing showing additional features of the humidifier 1701. The humidifier 1701 may include a child lock feature 1702 in order to lock the humidifier 1701 in a given state, such as on, off, or within a set operation protocol in order to prevent curious infants and toddlers from interfering with the operation of the humidifier. The humidifier water reservoir 1703 may be accessed from the top of the humidifier housing. The humidifier 1701 may include a scent diffuser 1704 and may include a separate chamber for receiving essential oils or other fragrances. The humidifier 1701 may include a UV-C sterilizing feature 1705. The humidifier 1701 may be adapted for use with custom filters 1706, including demineralization filters. The humidifier 1701 may have different preset modes with set humidity levels, such as normal mode, dry skin mode, and sick or illness mode for optimizing the nursery environment.

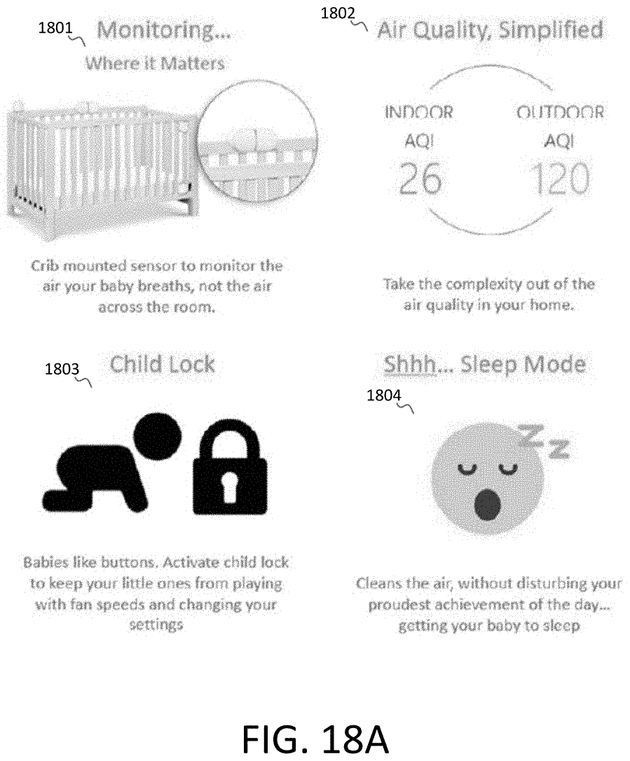

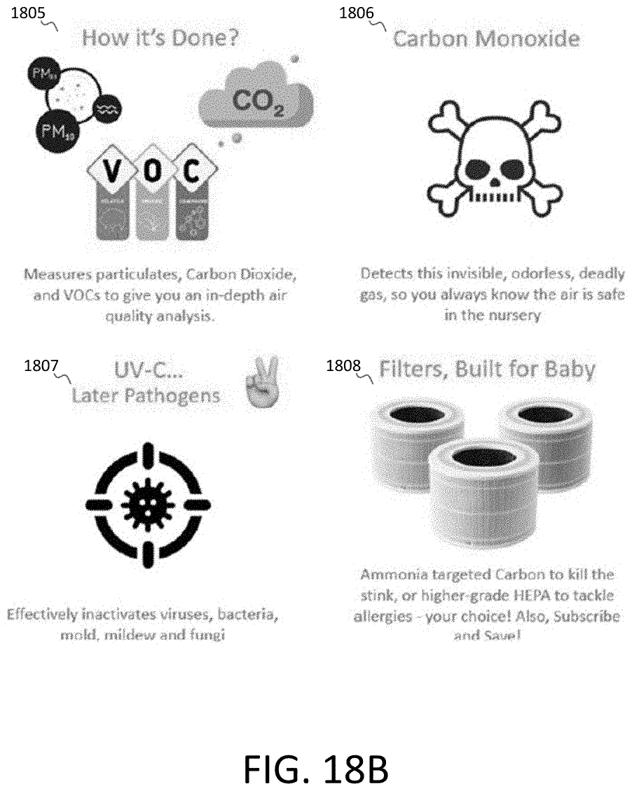

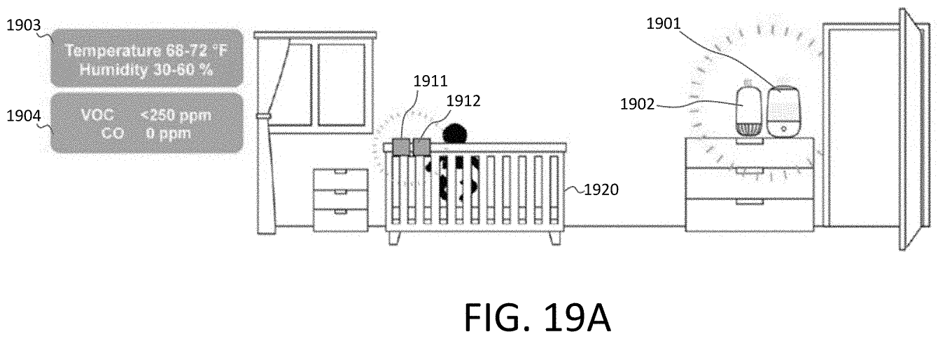

FIGS. 18A-18B are schematic drawings showing additional features of the air purifier, including a child lock 1803, a silent or sleep mode 1804, simple air quality reporting metrics 1801, color-coded Air Quality Index (AQI) alerts and infographics 1802, other informative graphics 1805, carbon monoxide detection 1806, UV-C purification 1807 and custom filters 1808. The air purifier may be set to a target AQI and may automatically adjust fan speeds to achieve the target goals. The air purifier may include a turbo mode to quickly correct unhealthy air, dangerous gases or contaminants, or adverse environmental conditions. Both the air purifier and the humidifier may include energy saver modes for conserving energy expenditures.

As shown in the schematic 1801 of FIG. 18A, the crib mounted sensor monitors the air the baby breathes, not the air across the room. The schematic 1802 shows air quality at the point of the sensor and can compare it with an outdoor air quality measure. The child lock feature 1803 keeps the infant or child from playing with fan speeds and changing settings. The schematic 1804 shows the air can be cleaned without disturbing the sleeping infant. In FIG. 18B, schematic 1805 shows that the air purifier can measure particulates, carbon dioxide, and VOCs to give an in-depth air quality analysis. Schematic 1806 shows the air purifier can detect carbon monoxide, the invisible, odorless, deadly gas, to ensure that that the air is safe in the room where the air is being monitored. The air purifier can effectively inactivate viruses, bacteria, mold, mildew, and fungi, as depicted in the schematic 1807. Finally, the air purifier can use various custom filters, including ammonia targeted carbon to kill bad unpleasant odors, or higher grade HEPA filters to tackle allergies.

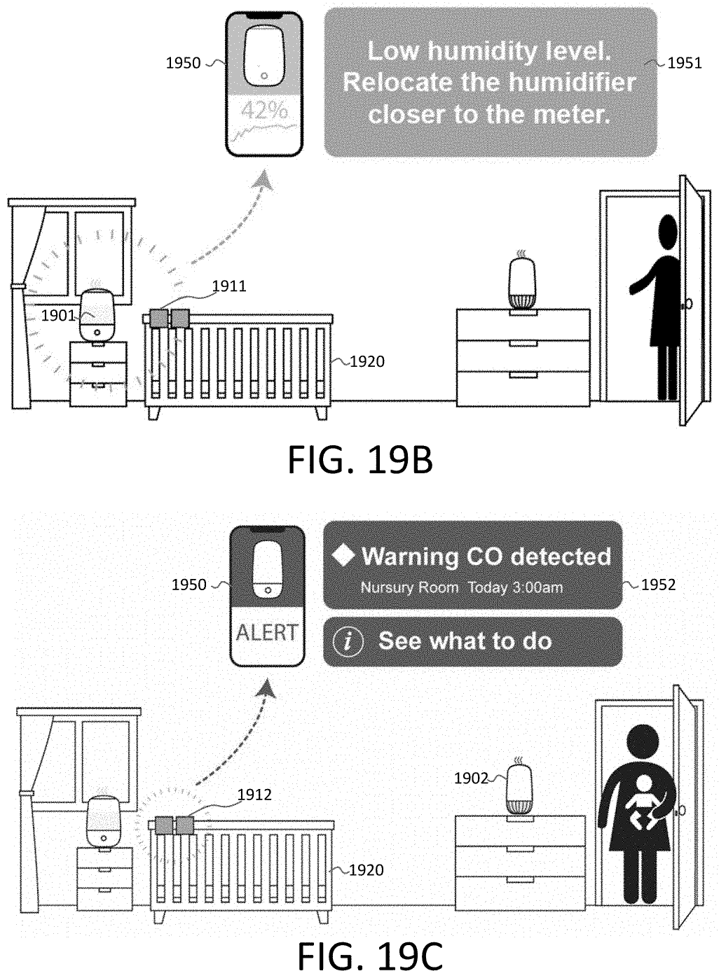

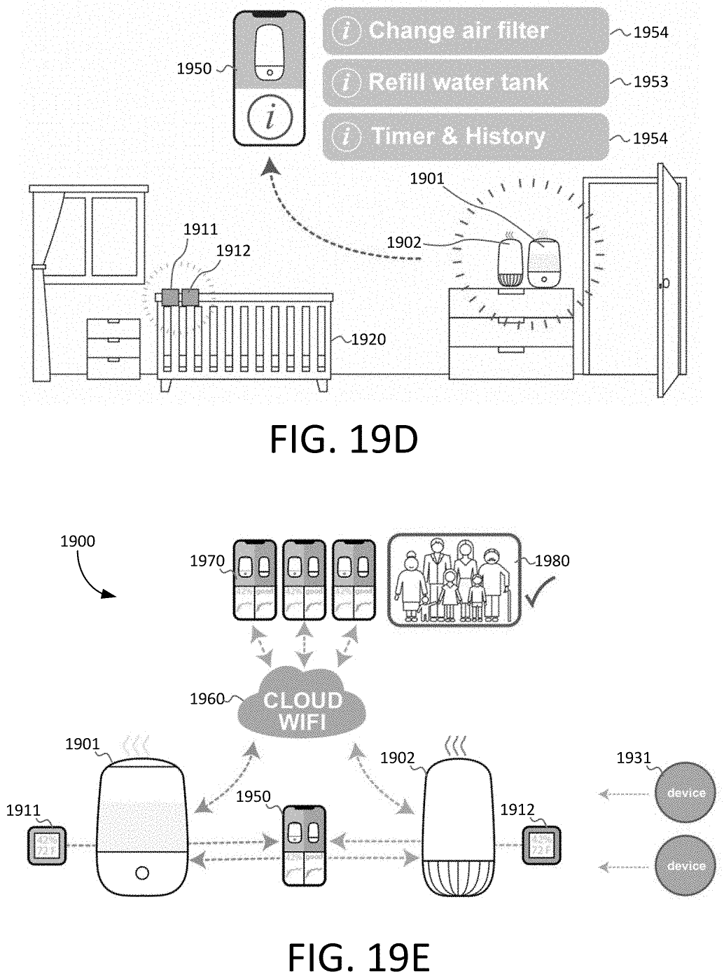

FIGS. 19A-19E illustrate a specific but non-limiting example of operation of the humidifier 1901 and air purifier 1902 with sensors 1911, 1912, respectively, in a nursery and a method of monitoring a nursery. The humidifier 1901 and air purifier 1902 are in constant communication with the sensors 1911, 1912, respectively, attached to the infant's crib 1920. The sensors 1911, 1912 constantly generate environmental data 1903, 1904, respectively, that is transmitted back to the humidifier 1901 and air purifier 1902 in order to adjust the operation of these devices to achieve the programed parameters set for the nursery. Because of their proximity to the infant, the sensors 1911, 1912 generate accurate readings of the infant's actual environmental conditions. As shown in FIGS. 19B-19E, the sensors 1911, 1912, and devices 1901, 1902 are also in communication with the monitor application 1950 in order to notify the caregiver of important events and information.

FIG. 19B shows the humidifier 1901 communicating to the monitor application 1950 that the humidity sensor 1911 has detected a low humidity level and the suggestion 1951 that the humidifier 1901 be moved closer to the crib 1920 and sensor 1911. Such information indicates that there is insufficient output due to environmental changes, and an informed decision and guidance to relocate the device or provide further water to humidify the room

FIG. 19C shows the air purifier sensor 1912 alerting the monitor app 1950 that harmful carbon monoxide has been detected. A phone alert 1952 is generated by the monitor app 1950 in order to notify the caregiver that the child is potentially in danger. Such information provides an alert that there are dangerous gases and an unsafe environment.

FIG. 19D shows the air purifier 1902 communicating to the monitor app 1950 important information 1954 such as the need to replace the air filter, and the humidifier 1901 communicating information 1953 that the water reservoir is low. The monitor app 1950 allows the user to set user preferences 1954 such as set timers, schedules and collect data and track user history.

FIG. 19E is a schematic diagram showing the IoT ecosystem 1900 is use. The smart devices 1901, 1902 are in communication with the sensors 1911, 1912, respectively, the monitor app 1950, and the cloud server 1960. The monitor app 1950 is in communication with the sensors 1911, 1912, devices 1901, 1902, 1931 and cloud server. The cloud server is in communication with the monitor app, sensors and devices. The monitor app 1950 may be utilized simultaneously on one or more than one smart device 1970 by more than one user 1980. The monitor app 1950 may allow users 1980 accessing the app on different smart devices 1970 to communicate and access the same or different user profiles. The monitor app 1950 may allow for different user profiles 1980 and unique child settings for different individual children. The IoT ecosystem 1900 allows for real-time monitoring, automatic device settings and informed, data-driven guidance and education for caregivers. The monitoring app 1950 may store sleep data for individual users and generative optimized predictive operational parameters based upon the collected data and user history. What the IoT ecosystem 1900 provides is the control from anywhere by multiple users via the cloud 1960, and the connectivity with multiple devices 1901, 1902, 1931 in the ecosystem 1900.

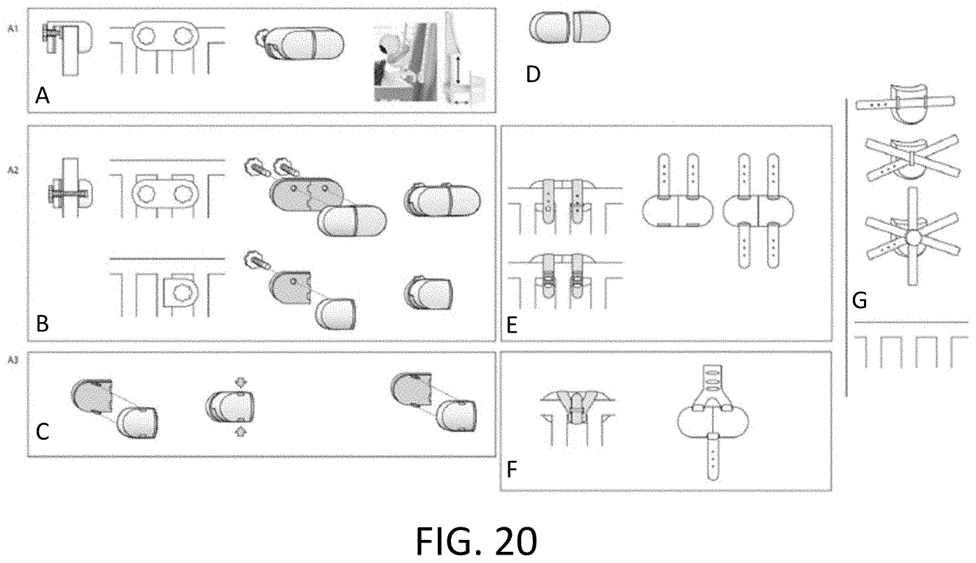

FIG. 20 shows different attachment mechanisms for attaching the sensors to a crib or other structures. Clamps, friction-fits screws, straps, compressible housings, magnet attachments, and mating front and back components are just some of the relevant features disclosed for attaching the sensors to an appropriate surface. Some non-limiting examples of attachment mechanisms for the sensors 1901, 1902 include: (A) top rail of crib with screw; (B) vertical posts with screw; (C) snap fit attachment, (D) magnetic attachment; (E) vertical strap attachment to the top rail of crib; (F) alternate belt-like vertical strap attachment to the top rail of crib; and (G) horizontal strap attachment. Other mechanisms and techniques are also possible and within the purview of the present subject disclosure.

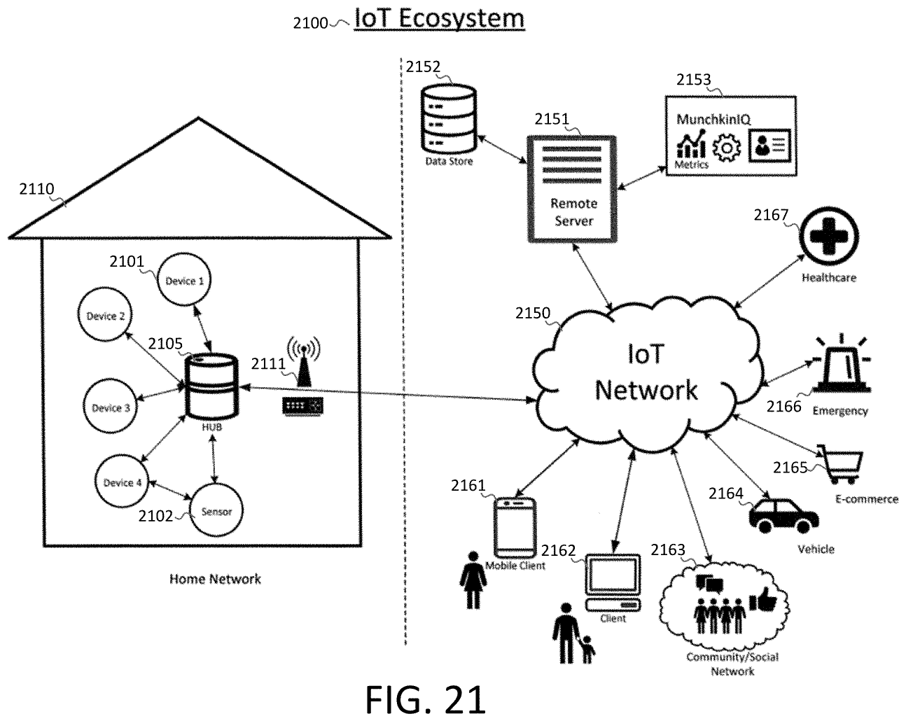

As shown in FIG. 21, the IoT ecosystem 2100 may include one or more devices 2101 in a home network 2110 or other location. The devices 2101 may be any of the above listed smart devices as described above or other suitable devices, and may be connected to an associated sensor 2102. The devices 2101 may be connected to an IoT hub 2105. The hub may be adapted to control the various devices 2101 included in the IoT ecosystem 2100. The hub may 2105 be integrated into one of the previously discussed smart devices, or the hub may be a standalone device. Alternatively, a home router 2111 may serve as a hub into which all of the devices 2101 in the home network 2110 are connected and communicate therefrom into the internet. The hub 2105 may be adapted to connect to an IoT network 2150 through a wired or wireless internet connection. The hub 2105 may have a processor, memory, a communication module and a locational unit having a global navigation satellite system (GNSS) receiver. The GNSS receiver may be a global positioning system (GPS) receiver. The processor may be electrically coupled to the memory, the communication module and the other electrical components attached therein by one or more high-speed buses.

The home network 2110 may be a bespoke network or a network adapted to run on common IoT platforms such as Google Cloud, IRI Voracity, Particle, Salesforce IoT Cloud, IBM Watson IoT, ThingWorx, Amazon AWS IoT Core, Microsoft Azure IoT Suite and the like. The IoT network 2150 may be a distributed network and may be partially or entirely substantiated on one or more remote servers 2151. The remote server 2151 may have a processor, memory and a server communication module coupled together by one or more high speed buses. The remote server 2151 may be connected to one or more database 2152. The database 2152 may be stored in the memory of the remote server, or may exist on a separate server, a virtual server or hosted in a cloud network. The remote server memory 2153 may run the MunchkinIQ application and its associated routines, algorithms, machine learning or artificial intelligence (AI) applications, data, software, logs, or a combination thereof. The MunchkinIQ application is a specific smart device program or stand alone software that is specifically designed to interact with the subject disclosure as described herein.

The IoT network 2150 may be connected to one or more client devices and one or more service providers. The client device may be a portable computing device 2161 such as a smartphone, a desktop, a laptop, a tablet, a smartwatch, an entertainment device, and/or a voice-enabled smart device such as a Google home device, Amazon Echo device, Apple smart device, Facebook smart device, Samsung smart device, LG smart device, Sonos smart device, or another smart home controller 2162 or hub device. The IoT network 2150 may be connected to one or more vehicles 2164 and/or one more in-vehicle computers. The IoT network 2150 may be connected to one or more service providers. The connected service provider may be a healthcare provider 2167, such as a doctor, a doctor's office, a hospital, or other health provider. The service provider may be one/or more emergency services 2166 such as the police or fire department. One or more community/social networks 2163 may be connected to the IoT network 2150. The social network 2163 may be a network or community of parents and other caregivers that share stories and child rearing advice. The community may share preferred settings for the various devices 2101, 2161, 2162, etc. based on the best practice learned with their children and the like. Childcare experts may be included in the social network and the network may feature informative blogs, recommendations, and articles for assisting caregivers. The IoT network 2150 may be connected to one or more Ecommerce platforms 2165, such as Munchkin, Amazon, Walmart and the like for facilitating the purchase of essential and/or desired products for assisting with childcare duties.

The IoT network 2150 may include any multi-hop network or wide area network (WAN) that covers cities, states, regions, countries or other locations. The IoT network 2150 may include any number of wireless local area networks (WLANS), including those established under IEEE's 802.11 protocol or its successors. The WLANS may include a number of wireless-fidelity (WiFI) networks. The IoT network 2150 may incorporate cellular networks such as 3G, 4G, 5G, long-term evolution (LTE) networks, the internet, a satellite network, a Starlink network

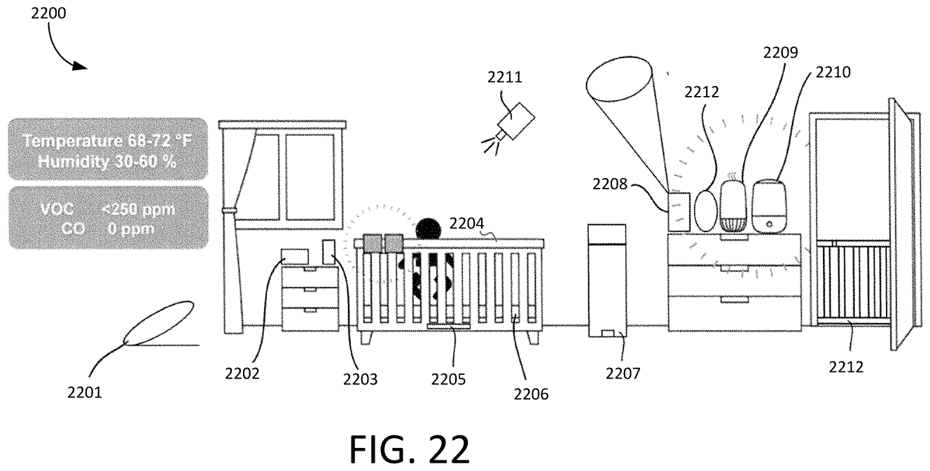

FIG. 22 illustrates a home network 2200 within the IoT ecosystem in a nursery, according to an exemplary embodiment of the present subject disclosure. The home network 2200 may include a bouncer 2201, rocker or swing, wipes warmer 2202, bottle warmer 2203, a smart crib 2204, vibration device 2205, pad with sensor 2206, diaper pail 2207, infant soother with light projector 2208, air purifier 2209, humidifier 2210, monitor 2211 that may capture audio and visual data, a smart gate 2212, a smart lock, various sensors and/or other smart home controllers, voice-assisted devices 2212 (GOOGLE, ECHO, ALEXA, etc.) or hub devices. The home network 2200 may include the IoT hub. The IoT hub may be incorporated into any of the above listed devices, a standalone device and/or a virtual cloud device in the IoT network. The IoT hub may be adapted for use in the nursery and/or use in another room in the house, such as the kitchen, living room, bathroom or a caregiver's bedroom. The home network 2200 may include other smart devices located in the house or environment.

The devices in the home network 2200 may be in wireless communication directly with the hub, connected client devices, sensors, and/or the connected devices through a short-range wireless communication protocol, such as a Bluetooth protocol, Bluetooth Low Energy (BLE) protocol, ZigBee protocol, near-field communication (NFC) protocol, any combination thereof, and/or any of the communication protocols previously discussed above.

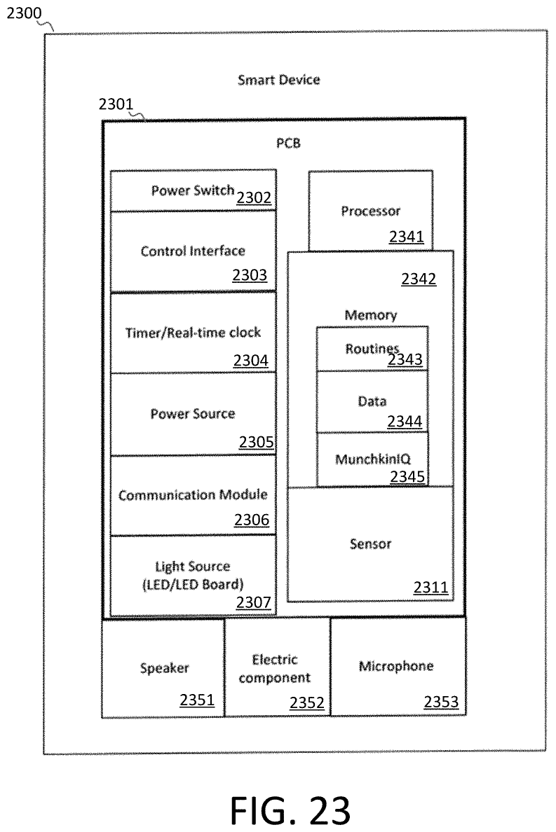

FIG. 23 details a block diagram of a common smart device 2300 printed circuit board (PCB) 2301 comprising a number of chips, modules, integrated circuits (ICs), sensors, interfaces and high-speed buses. For example, the PCB may include a power switch 2302, a user control interface 2303, a timer and real-time clock IC 2304, power source 2305, a communication module 2306, one or more sensors 2311, a processor 2341 and memory 2342. Other components 2352 may be electrically coupled to the PCB 2301 and incorporated into a device circuit. For example, a light source, a speaker 2351, a vibrating device, a microphone 2353, a digital clock, ambient lights, a capacitive touchscreen and/or other components may be incorporated into the device circuit. The light source may be one or more LEDs electrically coupled to an LED circuit board. The LED circuit board may be the PCB 2301 or a separate LED circuit board that is electrically coupled to the PCB. The LED board may be separated from the PCB 2301 by a gap or distance and may be positioned above or elevated from the PCB. The PCB 2301, the LED Board and the other integrated electrical components may be coupled to one another by clips, clasps, adhesives, fasteners, screws, thread connections, interference fit, heat staking, thermoplastic staking by laser welding or ultrasonic welding, or a combination thereof. The PCB 2301 or LED board may be attached to the device housing through conventional means such as studs, posts, ribs, bosses that extend from the housing and attach to the boards through holes, slots, staking, interference fit, or a combination thereof.

The device processor 2341 may execute software or routines 2343 stored in the memory 2342 to execute the methods described herein. The processor 2341 may be an embedded processor, a microprocessor, a logic circuit, a hardware finite-state machine (FSM), a digital signal processor (DSP), or a combination thereof. The processor 2341 may include one or more central processing units (CPUs), graphical processing units (GPUs), application-specific integrated circuits (ASICs), field-programmable gate arrays (FPGAs), or combination thereof.

The device memory 2342 may store software, AI or machine learning algorithms, routines, microcode, firmware and logs. The memory 2342 may be internal memory or external memory such as an external storage unit. The memory 2342 may be non-volatile memory such as read-only memory (ROM), NVRAM, Flash memory and/or disc storage. The memory may be volatile memory such as random-access memory (RAM), DRAM and/or SRAM. Data 2344 and the MunchkinIQ application 2345 may be included in the device memory 2342. The memory 2342 may be adapted to receive software and firmware updates from the IoT network.

The device communication module may include one or more wired or wireless communication interfaces. The communication module may be a network interface card of the smart device. The communication module may be a wired or wireless modem and may support WiFI, 3G, 4G, LTE, Bluetooth and/or radio technologies. The communication module may include a receiver and an antenna. The communication module may communicate with a wide area network (WAN) in order to transmit or receive packets, data, messages and/or instructions.

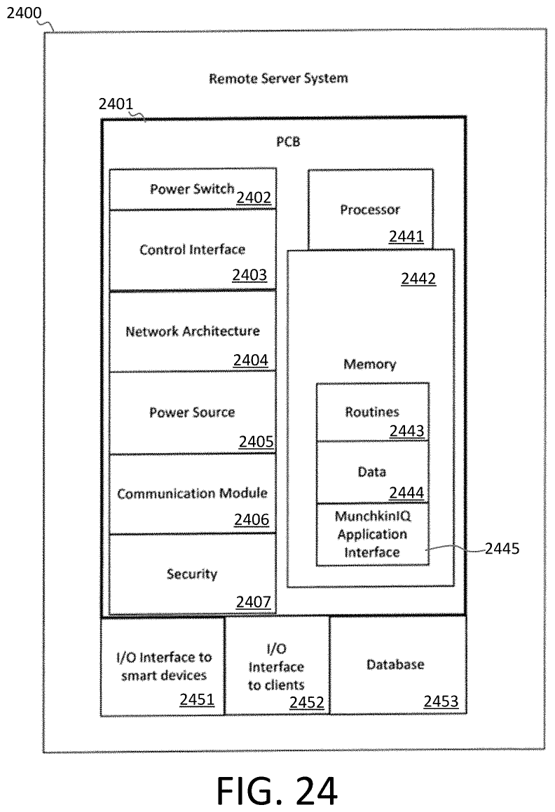

FIG. 24 is a block diagram drawing of a remote server system 2400. The server may have a processor 2441, memory 2442, a server communication module 2406, and/or other electrical components forming a circuit and in communication through high-speed buses.

The server processor 2441 may execute software or routines 2443 stored in the server memory 2442 to execute the methods described herein. The processor 2441 may be an embedded processor, a microprocessor, a logic circuit, a hardware finite-state machine (FSM), a digital signal processor (DSP), or a combination thereof. The processor 2441 may include one or more central processing units (CPUs), graphical processing units (GPUs), application-specific integrated circuits (ASICs), field-programmable gate arrays (FPGAs), or combination thereof.

The server memory 2442 may store software, routines 2443, logs, network architecture, communication protocols, encryption protocols, security protocols for secure communication through the IoT network and between the remote servers and endpoint devices, data 2444 for data processing protocols and instructions and other features and software associated with the MunchkinIQ application 2445 and network. The memory 2442 may be internal memory or external memory such as an external storage unit. The memory 2442 may be non-volatile memory such as read-only memory (ROM), NVRAM, Flash memory and/or disc storage. The memory 2442 may be volatile memory such as random-access memory (RAM), DRAM and/or SRAM. The memory 2442 may be adapted to send software and firmware updates to the various devices connected through the IoT network. The server memory 2442 may include or be connected to one or more databases for the storage and processing of data 2444 associated with the MunchkinIQ application 2445.

Further components on the PCB 2401 include but are limited to power switch 2402, control interface 2403, network architecture 2404, power source 2405, communication module 2406, security 2407, I/O interface to smart devices 2451, I/O interface to clients 242, and database 2453.

The server communication module 2406 may include one or more wired or wireless communication interfaces. The communication module may be a network interface card of the server. The communication module 2406 may be a wired or wireless modem and may support WiFI, 3G, 4G, LTE, Bluetooth, BLE and/or radio technologies. The communication module 2406 may include a receiver and an antenna. The communication module 2406 may communicate with a WAN, WLAN or other network in order to transmit or receive packets, data, messages and/or instructions through the server communication module.

The MunchkinIQ application 2445 may be adapted to execute one or more graphical user interface (GUI) on the client devices or other smart devices connected to the IoT network. The MunchkinIQ app may be stored in the memory 2442 of the remote serve, or the memory of the client devices or smart devices. The MunchkinIQ software may also be hosted in a cloud server. The MunchkinIQ GUIs may allow a user to interact with, and control, the MunchkinIQ application and related software.

The MunchkinIQ application may be adapted to collect data related to the IoT ecosystem, connected devices and various users of the application. The application may be adapted to generate suggestions, advice and education information for caregivers using the data collected, and/or other sources. The application may be adapted to identify a caregiver's problem or unique situation and generate advice or education related to sleep training, nutrition, helpful childcare products, growth and development milestones, and the like. The application may send push notifications to client devices and smart devices to provide advice, alerts and other notifications. The application may be linked to one or more ecommerce platforms in order to facilitate the purchase of necessary and desired products. The application may incorporate regional information such as weather and air quality index (AQI) and send notifications related to this information to the users. The application may include a user community that pools caregiver experiences, stories, advice, and other information related to their use with the IoT ecosystem and child care. For example, the user community may share personalized set-up configurations and best uses of the connected devices. The application may include a neighbor function for connecting with other caregivers in the area. The MunchkinIQ application may include various modes, for example, a sleep mode, a nap mode, a deep sleep mode, etc. The application may be adapted to detect, survey and share sales and marketing information with the user based on detected or learned information collected from analyzed data collected. The application may be adapted for use with multiple operating systems including Windows, iOS and Android.

The MunchkinIQ application may include security features to protect the user's privacy. The application may be adapted to control access to the home network and IoT ecosystem, the IoT network and the connected devices to specific family, friends, neighbors and babysitters. The application may be adapted for use with authentication, encryption and security software that may restrict access to the application and network based upon authorization, user identity, time restrictions, events, and the various modes of the application. The application may include a lock feature that locks the connected devices from use during certain modes or based upon user preference. In one embodiment, a lock feature may be provided to prevent a child from disabling the routine set by the caregiver.

The MunchkinIQ application may be adapted to turn on, shut off, power off or deactivate the connected smart devices. The MunchkinIQ application is adapted to provide GUIs that facilitate and actuate the user's ability to control the various features of the connected devices. The application may be adapted to record and update the GUI and application based upon user actions and inputs on the physical devices. The application may render a firmware update GUI to inform the user that the firmware of a certain connected device is being updated. Updates to the MunchkinIQ software or device firmware may be pushed to the client devise or smart devices to improve the functions of the application or device and/or introduce new software or device features.

The IoT Eco System, as described in detail above has a number of factors and influences. These include, but are not limited to, MunchkinIQ app which collects data, work, vehicle, Mobile App (phone, computer tablet, remote control), health care, family networks (sitters, nannies), non-Munchkin smart devices (AI algorithms), security (data share, upload, download), community (blog/best mode, setup, present, neighbor function), and home (nursery, parent's room).

The IoT Display and Other Product Tie-In's (BT/WiFi/Wireless/Wired) may include, but not limited to: Apple TV/Firestick, Google/Echo/Alexa, Emergency 911, Alarm—Phone, Emergency Contact List, and Bose.

The Munchkin IQ app functions to, but is not limited to: Gather data ID the problem, Give Advice, Education (Sleep, Nutrition, Products, Milkmakers, Partnership with other companies/products, Current and Future Products, Baby Development/Milestones), Push Notifications, E-commerce, Regional Environment Knowledge, Create Community and provide Recommendations—like Ring App (Blog/Best Mode/Setup, Preset mode, Upload/Download Neighbor Functions/Recommendations), Smart Tracking/Recommendations, Sales/Marketing.

Privacy considerations include but are not limited to: Controlled Access (To family/friends/neighbor/sitter), Visibility in Nursery/Camera, Smart Block—Time restrictions/Nursing time, Timed Alert, Authorization Code for access, and Safe List.

The method used to implement MunchkinIQ is loaded onto a software.

Various modes are considered including but not limited to: Motion Sensor, Interconnecting, Sick, Sleep/Pre-deep Sleep/Nap, Wake, Changing Diaper, Emergency (CO2, Fire), Easy Button—Override sleep mode, alerts to phone regardless.

Various devices may be connected including but not limited to: Phone/Tablet, Apple Watch/Google Watch, Air Purifier, Humidifier—Scents, Lullavibe—vibration, Light Projector/Soother, Bounce/Rocker/Swing, and Smart Wipe Warmer (Smart heated weight on stack of wipes—low energy, heats when wipes needed).

Another connected device is a 59S stainless steel Pail (Odor Control, Auto Open (motor+Sensor), Scent Diffuser—fresh scent when lid automatically opens, Bag monitoring/Sterilization Cycle, Tracking Types, times, size of Diaper—log wet/dirty diapers Log feature, Can combine with Diaper Detection features (Let consumer know it's time to change diaper bag, Let consumer know it's time to change baby's diaper, Activates UVC and Scent diffusion)).

Another connected device is a Smart Bottle Warmer that is: Auto Timed to turn on at feeding time/certain times during the evening Can be button activated to start warming, Insulated to cool, and then activated to warm with integrated heater when activated within minutes with live countdown, Amount consumed detected, Measures temperature of milk/water for preloaded formula, Push notification—prescheduled feeding time/finished warming, At home/on-the-go.

Another connected device is a Smart Crib/Pad with sensor/Smart Bassinet. The Smart Garment/Connected Textile includes: Heart Rate Monitor, Heat/Cool Temperature Adjust (i.e., mattress), Diaper Detection, Movement/Position Detection (roll over), Crib Mesh with antimicrobial element (blocks out virus/bacteria), Eco-Smart—smart recyclable materials. The Monitors include: Video Monitor (Two-way audio monitor, Pre-recorded sounds/songs), Heart rate/Wellness monitoring with/without wearable, Ambient Temperature/Humidity, Fever/Body Temperature, Infant Sounds/Cry detection, Baby measurement—Body height/head size, Health Care Provider—Auto send vital data (send data for doctor visit, and Organized data for doc visit (eating, pooping, teething, meds, sleeping, etc.). Further monitors include: Cry Detection/Analyzing, Cough Detection, Weight/Scale, Vibration/Motion (side to side, up and down, orbital) with Themed motion pairs with sounds/vibrations for unique soothing environments (i.e. car ride), Sound Soothing (Binaural Sounds—dual speaker built into bassinet, and Sound Cancelling—canceling environmental sounds). Still other monitors include: Ice Pack/Heat Pack, Air Purifier, Humidifier Scent Diffusion (Scheduled scent for morning/night routine, Mom/Dad scent captured for baby comfort).

There is also Bedtime Routine with Projector (Sunset, Moon, Sunrise with specific LED wavelength, Eclipse feature on Moon for visual transition before light turns off, Chromotherapy).

Other monitor features include Display Screen, and ability to be Wearable (Smart Swaddle with built in sensors (heartrate, rollover detection, temperature, diaper detection).

There are Binaural Speakers which include: Two speakers locate on either side of head with shifting frequency on either side to induce sleep, One product with 2 speakers—sound pointed outward (Mounted to top of any crib, Sound projects outward).

The Monitor/Display/Remote Access to Operate one or more items include: Temperature monitoring, AC Unit, Radiator, Smart Ceiling Fan, Cool/heat room, Darken Room (Auto Blinds), Phillips/Hue/Lights, Movement, and Sound.

Other connected devices may include: A Wipe Warmer can be used to Light/Warm/UV Sterilize, Safety Accessories such as Gates (lock/unlock, turn on lights, Night locking mode, Night motion sense light), and a Door monitor (at night/door to outside), Smart Crib (Smart Lounge/Motion/Weight Sensor), Baby Ipad/Display, and TheraBurpee™.

Another connected device, as described in detail above is a Sound Soother/Projector with: One Touch on/off/routine activation (Sequenced air motion gesture control, Motion control (i.e. brightness/volume)), Bedtime Routine with Projector (Sunset, Moon, Sunrise with specific LED wavelength, and Eclipse feature on Moon for visual transition before light turns off), Sound Soother (Meditation—breathing exercise synchronize with projection), Cry Detection (Automatically starts soothing sounds when cry is detected, Cry detection setting can be set at low, medium, high sensitivity, Time threshold for response); Cry Analyzing: Uncomfortable (wet/dirty diaper)—notification/recommendation to parent's phone, Angry—activates soothing, Tired/Sleepy—activates soothing Hungry—notification/recommendation to parent's phone); Cough Detection; Ambient Light; Voice Enabled activation/control (Google/Alexa, Voice control embedded); Scent Diffusion (Sleep/Wake scents); and Sound Soothing (Binaural Sounds—dual speaker (speakers on both sides of child)—Promotes sleep, and Sound Cancelling—canceling environmental sounds).

Another connected device is for Diaper Detection, which has the features: Attached to baby (wearable) or 2-3 meters from baby (tabletop); Wet and Solid detection/analysis (Light wetness, heavy wetness, Stool Analysis (color, consistency, odor)); Air Quality Monitor (PM Sensors, Indoor Air Quality, Outdoor Air Quality); Cry Detection (Know if your baby is crying due to a dirty diaper, and Helps with sleep training—no need to intervene if no diaper is detected); Humidity Monitor; Temperature Monitor; Co2 Monitor Smart Garment—all features above can be incorporated into wearable smart garment; Video monitor/Camera (Video monitors heat/cool to determine if a diaper has been soiled, Senses warmth of initial wet diaper, with cool down after diaper has been wet for a period of time, Camera can detect blue line through clothing—either IR or smart garment).

Another connected device is a Diaper Detection System, which has the features: Odor Control—Detects odor and activates Scent Diffuser; Air Quality Control—Detects low indoor air quality and activates Air Purifier; Humidity Control, and Temperature Control.

Another connected device is a Hub/Parent Monitor, which has One location for all Munchkin IOT system to report/display, and Displays hologram of baby on the Hub.

A sensor detects and activates various functions, for example Cry→Light, Soothing, Wipe Warmer, Bottle Warmer. A Temperature→Vibration.

A vehicle/Smart Vehicle includes the following: Mirror, Camera Sensor in mirror, Motion/Presence Sensor in mirror or vehicle, Left Baby in Car detection and notification, Heating/Cooling—Start A/C, Display Under Mirror, Start Kid Show—Car Sensor, Speaker embedded (Soother, White Noise, Works with Vibration), Health Vitals, Cry Sensor, and Air Purifier/Humidifier on the go.

Personal Soothing Devices can include: Baby Speakers surrounding head rest, Nest Child, Noise Cancelation Headphones.

Remote Diagnostics can include Heart Rate Monitor and Temperature.

A smart app can include Preferences, security, data tracking and measuring; Operates with Multiple mobile operating systems (iOS, Android, Windows); Products light up; Customizable routines; Routine Upload/download updates.

Feeding time can be made soothing with the present subject disclosure.

Parents Room can include Touch Pad—Alerts in parents' room; Lights, soother, humidifier, air purifier; Cross talk/Smart button; and Easy button.

Each of the individual variations or embodiments described and illustrated herein has discrete components and features with may be practiced separately or in combination with any of the other variations or embodiments. Modifications may be made to adapt to a specific situation, material, composition of matter, process, method or scope of the present invention.

Methods recited herein may be carried out in any order of the recited events that is logically possible. Moreover, additional steps and operations may be provided or steps or operations may be eliminated to achieve the desired results.

Furthermore, where a range of values is provided, every intervening value between the upper and lower limit of that range and any other stated or intervening value in the stated range is encompassed within the invention. Also, any optional feature of the inventive variations described may be set forth and claimed independently, or in combination with any one or more of the features described therein.

The illustrations and examples provided herein are for explanatory purposes and are not intended to limit the scope of the appended claims. It will be recognized by those skilled in the art that changes or modifications may be made to the above described embodiment without departing from the broad inventive concepts of the invention. It is understood therefore that the invention is not limited to the particular embodiments described herein, but is intended to cover all modifications and changes within the scope and spirit of the invention.

What is claimed:

1. An infant soother comprising:
a housing;
a speaker; and
a projector;
wherein the projector is adapted to project a first light configuration onto a surface and transition to at least a second light configuration while the speaker generates an accompanying sound.

2. The infant soother of claim 1, wherein the transition from the first light configuration to the second light configuration changes in color.

3. The infant soother of claim 1, wherein the transition from the first light configuration to the second light configuration changes in intensity.

4. The infant soother of claim 1, wherein the first light configuration and the second light configuration project from an aperture in the housing.

5. The infant soother of claim 1, wherein the first light configuration and the second light configuration project from a lens in the housing.

6. The infant soother of claim 1, further comprising an ambient light which projects from a translucent wall portion of the housing.

7. The infant soother of claim 1, further comprising a rotating disk which controls the timing and projection of the first light configuration and the second light configuration.

8. The infant soother of claim 7, wherein intensity and duration of the first light configuration and the second light configuration are manually controllable through an external dial.

9. The infant soother of claim 1, further comprising a manually controllable aperture in the housing to control the size of the first light configuration and the second light configuration.

10. The infant soother of claim 1, further comprising a keystone control feature which controls the even projection of the first light configuration and the second light configuration.

11. A smart nursery, comprising:
an air modification device;
a sensor that is separate from but wirelessly connected to the air modification device; and
an electronic device which stores a software application that controls and communicates with the sensor and/or the air modification device.

12. The smart nursery of claim 11, wherein the air modification device is a humidifier.

13. The smart nursery of claim 12, wherein the sensor detects humidity in the air and communicates a measured value to the humidifier.

14. The smart nursery of claim 13, wherein the humidifier initiates operation if the measured value of the humidity is less than a pre-determined value which has been programed in the software application in the electronic device.

15. The smart nursery of claim 11, wherein the air modification device is an air purifier.

16. The smart nursery of claim 15, wherein the sensor detects particulates in the air and communicates a measured value to the air purifier.

17. The smart nursery of claim 16, wherein the air purifier initiates operation if the measured value of any given particulate is greater than a pre-determined value which has been programed in the software application in the electronic device.

18. The smart nursery of claim 16, wherein the air purifier sends an alert message to the electronic device if a measured value of any given particulate is greater than a pre-determined value which has been programed in the software application in the electronic device.

19. A smart nursery, comprising:
one or more devices adapted to communicate electronically;
one or more sensors that are separate from but electronically connected to at least one of the devices;
an electronic device storing an electronic application which communicates with at least one the devices or sensors; and
a remote server system in communication with the electronic device and containing historical and operational information relating to the operation of the devices or the sensors.

20. The smart nursery of claim 19, wherein feedback from the remote server affects operation of the one or more devices.
